(19) United States
(12) Patent Application Publication
(10) Pub. No.: US 2009/0012115 A1
(43) Pub. Date: Jan. 8, 2009

(54) USE OF TP MODULATORS FOR THE TREATMENT OF CARDIOVASCULAR DISORDERS IN ASPIRIN SENSITIVE AND OTHER POPULATIONS

(75) Inventors: David R. Phillips, San Mateo, CA (US); Patrick Andre, San Mateo, CA (US); Charles J. Homcy, San Francisco, CA (US)

Correspondence Address: SEED INTELLECTUAL PROPERTY LAW GROUP PLLC, 701 FIFTH AVE, SUITE 5400, SEATTLE, WA, 98104, US

(73) Assignee: Portola Pharmaceuticals, Inc., South San Francisco, CA (US)

(21) Appl. No.: 12/114,642

(22) Filed: May 2, 2008

Related U.S. Application Data
(60) Provisional application No. 60/915,784, filed on May 3, 2007.
(60) Provisional application No. 60/915,785, filed on May 3, 2007.
(60) Provisional application No. 60/947,316, filed on Jun. 29, 2007.
(60) Provisional application No. 60/947,289, filed on Jun. 29, 2007.

(51) Int. Cl. A61K 31/4365; A61K 31/422; A61K 31/4406; A61K 31/18; A61K 31/444; A61K 31/192; A61K 31/403; A61P 9/00

(52) U.S. Cl. 514/301; 514/374; 514/357; 514/604; 514/332; 514/570; 514/411; 514/603

(57) ABSTRACT

The present invention provides methods and compositions useful in the treatment or prevention of cardiovascular disorders in individuals for whom therapy with a COX-1 enzyme inhibitor is not feasible due to sensitivity, intolerance, or resistance to the inhibitor. Additionally, the invention provides methods of treating cardiovascular disorders in an individual who is receiving a therapeutically effective dose of a TP modulator and is instructed or advised to avoid and/or not to take aspirin or another COX-1 inhibitor.

### CROSS-REFERENCES TO RELATED APPLICATIONS

This application claims the benefit under 35 U.S.C. § 119(e) of U.S. Provisional Patent Application No. 60/915,784, filed May 3, 2007; U.S. Provisional Patent Application No. 60/915,785, filed May 3, 2007; U.S. Provisional Patent Application No. 60/947,316, filed Jun. 29, 2007, and U.S. Provisional Patent Application No. 60/947,289, filed Jun. 29, 2007, where these (four) provisional applications are incorporated herein by reference in their entireties.

### BACKGROUND

1. Technical Field

The present invention relates to methods of treating or preventing thrombosis and other cardiovascular diseases and disorders in aspirin sensitive and other populations, using antithrombotic agents such as thromboxane receptor inhibitors.

2. Description of the Related Art

Arterial thrombosis causes acute myocardial infarction and thrombotic stroke, and is a major contributor to morbidity and mortality in the Western world. The role of platelets in arterial thrombosis is well established, since arterial thrombi are composed primarily of platelets, and antiplatelet drugs are effective in reducing the incidence of acute myocardial infarction and thrombotic stroke. Platelets play a pivotal role not only in the formation of arterial thrombosis, but also in the progression of atherosclerotic disease itself. Platelet involvement in the progression of atherosclerosis is a more recent finding that evolved from the recognition that atherosclerotic disease is a response to inflammation and that inflammatory mediators released from platelet thrombi (e.g., sCD40L, RANTES, TGFα, PF4, PDGF) are potent contributors to the development of atherosclerotic lesions (see, Huo Y., et al., Nat Med 9:61-67 (2003); Massberg S., et al., J. Exp. Med. 196:887-896 (2002); Burger P. C., et al., Blood 101:2661-2666 (2003)).

Mechanisms responsible for platelet thrombosis have been identified. Platelet adhesion under arterial shear rates is mediated primarily by collagen, which recruits von Willebrand factor from plasma, which in turn is recognized by platelet membrane GP Ib-V-IX, triggering the recruitment of the platelets at the site of vascular injury. Platelets also bind directly to collagen via two collagen receptors on platelets, the integrin α₂β₁, and the immunoglobulin-containing collagen receptor, GP VI. Platelet activation is initially mediated by the primary agonists, collagen during platelet adhesion, and thrombin, a protease generated in response to tissue factor (TF) exposed at sites of vascular lesions and also by the engagement of GP IIb-IIIa by different ligands (e.g., fibrinogen, vWF, and CD40L). In addition, several secondary agonists released from activated platelets function in autocrine loops to potentiate platelet activation. One is thromboxane A₂ (TXA₂), a product of the prostanoid pathway, which is initiated by the release of arachidonic acid from phospholipids in response to the primary platelet agonists. Released arachidonate is sequentially modified by COX-1, a platelet enzyme, yielding prostaglandin H₂ (PGH₂), a substrate for the widely distributed thromboxane synthase, which produces thromboxane A₂ (TXA₂). Both products, PGH₂ and TXA₂, are potent platelet agonists that induce platelet activation by binding to the TXA2 receptor, also known as TP. Another secondary agonist is adenosine diphosphate (ADP), which is released from platelet dense bodies upon platelet activation. ADP binds to two G-protein coupled receptors, P₂Y₁ and P₂Y₁₂. Additional secondary mediators include Gas6 and CD40L.

The primary antiplatelet drug used for regulation of platelet function in patients with cardiovascular disease is aspirin. The extensive use of aspirin is based on hundreds of randomized clinical trials that show a reduction in adverse events by 20-25% (BMJ 324:71-86 (2002)). One of the initial studies was the Second International Study of Infarct Survival (ISIS-2), a randomized trial of intravenous streptokinase, oral aspirin, both, or neither, among 17,187 cases of suspected acute myocardial infarction (ISIS-2 Collaborative Group, J. Am. Coll. Cardiol. 12:3A-13A (1988) and the ISIS-2 Collaborative Group, BMJ 316:1337-1343 (1998)). Subsequently, numerous trials, summarized in the Antithrombotic Trialist's Collaboration, BMJ 324:71-86 (2002), extended these observations, showing, overall, a 22% reduction in vascular events. Additional examples include the Primary Prevention Project (de Gaetano G., et al., Lancet 357:89-95 (2001)), where aspirin use (100 mg/d) significantly reduced the incidence of cardiovascular death from 1.4% to 0.8% in at-risk patients. In the recently completed HOT trial, hypertensive patients were randomized to low dose aspirin (75 mg/d) or placebo (see, HOT Study Group, Lancet 351:1755-1762 (1998)). The low dose aspirin regimen resulted in a 15% reduction in cardiovascular events and a 36% reduction in myocardial infarction. These data clearly show that aspirin effectively reduces morbidity and mortality by 20-25% in the at-risk patient population (see, Patrono C., et al., Chest 126:234 S-264S (2004)).

Clopidogrel (a thienopyridine) is the second most widely used antiplatelet drug. It is a prodrug that requires hepatic metabolism to generate an active metabolite that irreversibly inactivates the ADP receptor P2Y₁₂. While clopidogrel was shown more efficacious than aspirin in the CAPRIE trial (see, Lancet 348:1329-39 (1996)), the subsequent CURE trial (see, Yusuf S., et al., NEJM 345:494-502 (2001)) established that combining clopidogrel with aspirin conferred a 20% relative risk reduction vs placebo plus aspirin to patients with unstable angina or non-ST segment elevation Ml. The PCI-CURE sub-study demonstrated that this benefit extended to patients undergoing percutaneous intervention (PCI) (see, Mehta, et al., Lancet 358:527-33 (2001)).

The pharmacologic action of aspirin in inhibiting thrombosis is primarily due to the inhibition of prostaglandin H (PGH) synthase 1 (COX-1) in platelets (FIG. 1). Following platelet activation, COX-1 functions first as an oxidase to oxidize arachidonic acid (released from phospholipids) to prostaglandin G₂ (PGG₂) and second, as a peroxidase to generate prostaglandin H₂ (PGH₂). PGH₂ is metabolized by at least four enzymes to produce thromboxane A₂ (TXA₂), a platelet agonist and several prostaglandins: prostaglandin D₂ (PGD₂), an inhibitor of platelet function; prostaglandin E₂ (PGE₂), which presents a dual activity on platelet function; and prostaglandin F₂α (PGF₂α), an additional metabolite.

Aspirin diffuses through cell membranes, binds first to an arginine residue (120) and then functions by irreversibly acetylating COX-1 at the active site (Ser-529). Because COX-1 inhibition is irreversible, the antiplatelet activity of aspirin lasts through the platelet life span. Aspirin's optimal antithrombotic activity requires over 90% inhibition of TXA₂ synthesis, and is obtained at doses ranging between 75 and 320 mg/day (see, BMJ 308:81-106 (2004); and Patrono C., NEJM 330:1287-94 (1994)). Aspirin also inhibits COX-2 via acetylation of Serine residue 516 (FIG. 1). COX-2 is the inducible form of the enzyme that is responsible for the production of most of the prostaglandins in inflammation and cancer. Inhibition of the cardiovascular protective effects of vascular PGI₂ by aspirin has long been thought to function as a possible brake to the protective effects of aspirin. For example, the acetylsalicylic acid and carotid endarterectomy trial which studied 3000 patients scheduled to undergo catotid endarterectomy showed that the combined rate of stroke, Ml or death at 3 months was significantly lower in the low-dose groups than in the high-dose groups of aspirin (Taylor, et al., Lancet 1999; 353:2179-84).

Several pharmacokinetic and pharmacodynamic characteristics inherent to aspirin confer its net antithrombotic efficacy. A short half-life (˜20 minutes in the human circulation) that prevents effects on the overall vascular tree; a higher selectivity toward COX-1 than COX-2 (by approximately 100 fold); and the fact that COX-2 turns over in vascular and inflammatory cells occurs within hours while COX-1 cannot be regenerated into the circulating platelets.

Although the success of aspirin is remarkable, it has become apparent that some individuals do not benefit from aspirin therapy, while others cannot benefit from its protective effects. The first group relates to the so called “aspirin resistance” phenomenon, describing thrombotic events developing despite aspirin therapy. Emerging data indicate that the antithrombotic responses to aspirin and clopidogrel are variable, and may include non responders. Aspirin resistance has been associated with the inability of aspirin to either inhibit TXB₂ levels (marker of TXA2 biosynthesis) or effect in vitro tests of platelet function (i.e., light transmittance aggregometry in platelet rich plasma or whole blood, RPFA (Ultegra Rapid Platelet Function Assay), Platelet Function Analyzer, PFA-100, and thromboelastograph). A more accurate definition of aspirin resistance should relates to its inability to prevent thrombotic events.

Several hypotheses exist to explain the thrombotic events in patients on aspirin therapy. The first, and perhaps the most commonly accepted is the existence of a multitude of platelet agonists that function in a prostaglandin-independent pathway. The second is based on reports showing a COX-2-dependent TXA₂ synthesis, despite optimal COX-1 inhibition by aspirin (see, Vejar, M., et al., Circulation 81(1 Suppl): 14-11 (1990)). In accordance with this finding, platelet turnover in coronary artery bypass graft (CABG) patients is suspected to trigger aspirin resistance as newly formed platelets that express COX-2 (see, Rocca B., et al., PNAS 99:7634-9 (2002)) maybe less sensitive to inhibition by aspirin (see, Zimmermann N., et al., Circulation 108:542-7 (2003)). In the same study, the authors demonstrated additional antiaggregatory activities of a mixed thromboxane synthase and receptor antagonist (terbogrel) in aspirin-treated individuals, demonstrating the existence of a COX-1-independent TXA₂ synthesis. Another potential cause of aspirin resistance has been described by Catella-Lawson who showed that concomitant administration of reversible inhibitors of COX-1 (i.e., ibuprofen) reduce the antiaggregatory activity of aspirin (see, Catella-Lawson F., et al., NEJM 345:1809-17 (2001)).

The second group relates to individuals at-risk of cardiovascular thrombotic events who are aspirin sensitive and, thus, cannot avail themselves of the cardiovascular protection provided by aspirin.

The broad role of COX-1 in various homeostatic systems is at the origin of many of the side effects attributed to aspirin. Indeed, COX-1 is essential for platelet aggregation, but also for the integrity of the gastric mucosa, the kidney water-salt balance, and normal vascular tone.

The most common adverse effect of aspirin is a substantial increase in upper gastrointestinal bleeding (see, Patrono, et al., Chest 126:234 S-264S (2004)). This side effect is attributed to both the inhibition of the pro-aggregatory activity of TXA₂ and a reduced cytoprotection of the gastrointestinal mucosa mediated by decreased levels of PGE₂ and PGI₂. Although the use of a proton pump inhibitor has demonstrated some levels of efficacy in reducing the risks of bleeding (see, Lanas A., et al., NEJM 343:834-9 (2000)), there has been no clinical evaluation of the protective effects of anti-secretory agents in coronary artery disease (CAD) patients with cardiovascular diseases who require daily doses (75-325 mg) of aspirin. In addition, it is estimated that 1 out of 10 individuals taking 75-325 mg doses of aspirin will develop gastroduodenal ulcers (see, Yeomans N. D., et al., Aliment. Pharmacol. Ther. 22:795-801 (2005)).

Another severe side effect of aspirin relates to aspirin intolerance. Aspirin-exacerbated respiratory tract disease (with a prevalence of approximately 10%), urticaria/angioedema (<0.5%), and more rarely systemic sensitivity (anaphylaxis) have been reported in the general population. Urticaria/angioedema upon aspirin or NSAID challenge can occur in patients with chronic idiopathic urticaria. Increased levels of leukotrienes are suspected to enhance vasopermeability and induce urticaria (see, Grattan C. E., et al., Clin. Exp. Dermatol. 28:123-7 (2003)). Urticaria/angioedema can also occur in patients without idiopathic urticaria history, after challenge with one or more NSAID, and is believed to be triggered by the production of drug-specific IgE antibodies against the NSAID. In rare cases, NSAID can provoke anaphylaxis in an IgE-dependent manner. But the most common side effect of aspirin in aspirin sensitivity relates to rhinitis and asthma. Oral challenge with aspirin or NSAID indicated that 5-20% of asthmatic adults develop severe bronchoconstriction, and are, therefore, intolerant to these therapies. Consequently, aspirin and NSAIDs are contraindicated for asthmatics (see, Spector S. L., et al., J. Allergy Clin. Immunol. 64:500-506 (1979) and Stevenson et al., In Allergy: Principles and Practice, Rosby Yearbook, Inc., St. Louis Mo., 1747-65 (1993)).

Inhibition of COX-1 is believed to be the direct cause of the aspirin (and other NSAIDs)-induced asthma attacks (FIG. 2). COX-1 inhibition redirects arachidonic acid metabolism towards an increased synthesis of leukotrienes (LTs). Leukotriene metabolites involved in aspirin (or other NSAID acting on COX-1) intolerance include LTB₄ (in neutrophil chemotaxis and activation), LTC₄, LTD₄, and LTE₄, all known to mediate broncho and vasoconstriction, and to increase vascular permeability and eosinophil chemotaxis. Most of the pro-inflammatory actions of the cysteinyl leukotrienes derived from their binding to the CysLT₁ receptor (see, Sousa A. R., et al., N. Engl. J. Med. 347:1493-1499 (2002)).

The mechanism by which aspirin raises LT levels is attributed to the blockage of PGE₂ synthesis (see, Pavord I. D., et al., Lancet 345:436-438 (1995)). PGE₂ is an endogenous inhibitor of both 5-lipoxygenase activating protein (FLAP) and 5-lipoxygenase. Consequently, decreased PGE₂ levels enhance synthesis of LTs and histamine release from mast cells (see, Szczeklik A., et al., J. Allergy Clin. Immunol. 111: 913-921 (2003)), human eosinophils (see, Docherty J. C., et al., Biochem. Biophys. Res. Commun. 148:534-8 (1987)), and neutrophils (see, Tenor H. A., et al., Br. J. Pharmacol. 118:1727-35 (1996)). However, the sole inhibition of PGE₂ does not account for the asthma attacks per se, as fluid levels of PGE₂ do not differ between aspirin-tolerant and aspirin-intolerant asthmatics (see, Szczeklik A., et al., Am. J. Resp. Crit. Care Med. 154:1608-1614 (1996)).

Other mechanisms responsible for the intolerance reaction exist and some have been described. An overrepresentation of cells (mostly eosinophils) expressing LTC₄ synthase (the enzyme forming LTC₄, the precursor of both LTD₄ and LTE₄) has been shown in the bronchial biopsies of aspirin-intolerant asthmatic when compared with aspirin tolerant asthmatics, while similar levels of expression of COX-1, COX-2,5-LO, FLAP and LTA₄ hydrolase were reported in both tolerant and intolerant asthmatics (see, Cowburn A. S., et al., J. Clin. Invest. 101:834-846 (1998)). Whether the presence of such cells was at the origin or a consequence of the intolerance was not established. Increased number of nasal inflammatory leukocytes expressing CysLT1 (the cysteinyl leukotriene receptor) were also described in aspirin-sensitive patients with chronic rhinosinusitis (see, Sousa A. R., et al., N. Engl. J. Med. 347:1493-1499 (2002)). Finally, genetic polymorphisms have been found on the 5-LO gene that may relate to aspirin-intolerance (see, Kim S. H., et al., J. Korean Med. Sci. 20:1017-22 (2005)). However, more work will be required to determine whether genetic polymorphisms correlate with intolerance.

Several methodologies have been used successfully to treat aspirin-induced asthma attacks. Cysteinyl-leukotriene synthesis inhibitors and selective antagonists of the cys-LT receptor have demonstrated marked attenuation of the aspirin-induced respiratory reactions (see, Holgate S. T., J. Allergy Clin. Immunol. 38:1-13 (1996); Israel, Am. Rev. Respir. Dis. 148:1447-1451 (1993); Nasser et al., Thorax 49:749-56 (1994); Christie et al., Am. Rev. Respir. Dis. 143:1025-29 (1991); Dahlen et al., Eur. Resp. J. 6:1018-26 (1993); and Yamamoto et al., Am. J. Respir. Crit. Care Med. 150:254-7 (1994)). Others demonstrated significant protection in aspirin-intolerant asthmatics by inhalation of PGE₂ (Sestini et al., Am. J. Crit. Care Med 153:572-5 (1996)) before aspirin therapy.

The most common approach to treat aspirin-intolerance is aspirin-desensitization. Acetylsalicylic desensitization refers to the elimination of pharmacological and immunologic reactions by first stopping the aspirin therapy, then slowly increasing the exposure to oral acetylsalicylic acid. This methodology has been shown to reduce all pro-inflammatory markers involved in the aspirin sensitivity reaction linked to respiratory tract reaction (see, Namazy J. A., Simon R. A., Ann. Allergy Asthma Immunol. 89:542-50 (2002); reduced production of leukotrienes, downregulation of the cys-LT1 receptor for leukotrienes, and decreased levels of histamine).

As outlined previously, acetylsalicylic acid desensitization therapies are available for the general population. However, physicians rarely use this therapy in cardiovascular disease (CAD) patients with known aspirin sensitivity for many reasons. First, it necessitates a careful mechanistic approach and the involvement of both cardiologist and allergist. Second, the safety of aspirin desensitization has never been investigated in patients with CAD. Indeed, both the American College of Cardiology and the American Heart Association guidelines indicated a class I indication for aspirin therapy in patients with coronary heart diseases and myocardial infarction (see, Braunwald, et al., Circulation 102:1193-1203 (2000); Ryan et al., Circulation 100:1016-30 (1999)), unless a true sensitivity to aspirin or NSAID was reported. The substitutive therapy recommended in these patients was the use of a thienopyridine (either clopidogrel or ticlopidine). However, the results of the CURE and CREDO trials lead to a new class I indication for the combination of aspirin plus clopidogrel for patients with unstable Angina/non-Q-wave myocardial infarction (see, Braunwald et al., J. Am. Coll. Cardiol. 36:990-1062 (2000)). This combination therapy is also indicated in order to reduce the risk of acute and subacute stent thrombosis (see, Mehta et al., Lancet 358:527-33 (2001); Moses J. W., et al., NEJM 349:1315-23 (2003); and Morice M. C., et al., NEJM 346:1773-80 (2002)).

One might ask, therefore, if it is possible for the aspirin intolerant population to use alternative drugs that mimic the cardiovascular protection provided by aspirin, but do not initiate the inflammatory reactions inherent to aspirin. Despite a long-felt need, this question remains difficult to answer. Indeed, the existing clinical studies of anti-thrombotic agents commonly exclude aspirin sensitive individuals from study.

TXA₂, the prothrombotic product resulting from the action of COX-1, activates platelets by acting on the TXA₂ receptor, also known as TP. Ex vivo experiments have shown that aspirin and TP antagonists inhibit platelet stimulatory events induced by platelet agonists such as collagen, and experiments in animal models have shown that TP antagonists are as effective as aspirin in blocking arterial thrombosis. The data indicate, therefore, that TXA₂ is the prothrombotic mediator blocked by aspirin and that TP antagonism provides an alternative strategy for blocking the action of this prothrombotic mediator. However, in view of their closely related modes of action, the suitability of TP antagonist therapy for treating or preventing cerebrovascular and cardiovascular thrombi in the aspirin-sensitive population remains unknown and uncertain.

The discovery and development of TXA₂ receptor antagonists has been an objective of many pharmaceutical companies for approximately 30 years (see, Dogne J-M, et al., Exp. Opin. Ther. Patents 11: 1663-1675 (2001)). The compounds identified by these companies, either with or without concominant TXA₂ synthase inhibitory activity, include ifetroban (BMS), ridogrel (Janssen), terbogrel (BI), UK-147535 (Pfizer), GR 32191 (Glaxo), and S-18886 (Servier). Preclinical pharmacology has established that this class of compounds has effective antithrombotic activity obtained by inhibition of the thromboxane pathway. These compounds also prevent vasoconstriction induced by TXA₂ and other prostanoids that act on the TXA₂ receptor within the vascular bed. The pharmacokinetic properties of several of these compounds in man is consistent with once-a-day dosing (see, Samara E., Cardiovasc. Drug Rev. 14: 272-285 (1996) and Liao W, et al., Clin. Pharmacol. Ther. 55: 2 (1994)). Although some of these compounds have specific issues, overall, this class of compounds appears to be safe with regard to their minimal effects on gastrointestinal bleeding.

Unfortunately, however, the Phase II/III trials in the US for TXA₂ antagonists have not proven successful. Accordingly, none of these compounds have reached the marketplace. In the CARPORT trial, GR 32191 was found to lack activity in the prevention of restenosis (see, Serruys P. W., et al., Circulation 84:1568-1580 (1991)). In the RAPT (Ridogrel vs. Aspirin Patency Trial) study, 907 patients suffering from acute myocardial infarction were randomized to receive either aspirin or ridogrel in addition to streptokinase. Ridogrel was not found to be superior to aspirin in enhancing fibrinolytic efficacy of streptokinase, although the study concluded that ridogrel may have been more efficacious than aspirin in preventing new ischaemic events (The RAPT Investigators, Circulation 89:588-595 (1994)). Sulotroban was studied in 752 patients in the M-HEART-II study on late clinical outcomes and restenosis following PTCA. Sulotroban was found to be no different than aspirin or placebo on restenosis and was inferior to aspirin in reducing clinical events defined as the combined endpoint of death, MI or clinically important restenosis (see, Savage M. P., et al., Circulation 92:3194-3200 (1995)). Nevertheless, it is commonly accepted that a poor choice of the clinical indications mainly accounted for the apparent lack of efficacy of this drug class. This was confirmed by the DAVID study which demonstrated that Picotamide (a dual thromboxane synthase and receptor antagonist) was significantly more effective than aspirin in reducing mortality in type 2 diabetic patients with peripheral arterial disease (see, Neri Serneri, European Heart Journal 25:1845-52 (2004)).

For many years, it was believed that the function of ADP and TXA₂ in platelet aggregation was to cause further platelet activation and recruitment of circulating platelets to the site of injury, while maintaining the activation state of GP IIb-IIIa. Experimental evidence suggests that the clinical efficacy of the state-of-the art antiplatelet therapy (clopidogrel+aspirin) utilized in the management of thrombotic disorders stems from a synergism in destabilization activities of the two drugs. Indeed, in vivo animal models of thrombosis have indicated that the primary functions of ADP and TXA₂ may relate in fact to thrombus stability and not to thrombus growth. Although the contribution of TXA₂ to the cohesion of arterial thrombi was reported twenty years ago in a dog coronary thrombosis model (Fitzgerald, D. J., et al., J. Clin. Invest. 77:496-502 (1986)), its relative role in the kinetics of human arterial thrombosis remains poorly understood.

Seratrodast, a TXA₂ receptor antagonist, and ozagrel, a thromboxane synthase inhibitor, are now marketed as anti-asthmatic drugs in Japan. Seratrodast and ramatroban, a thromboxane receptor antagonist, are in clinical trials in the US for the same indication. The suitability for these agents with regard to aspirin sensitive individuals is also not yet established. TP modulators may work in part by blocking the action of PGD₂ in promoting bronchoconstriction (see, Johnston et al., Eur. Resp. J. 8:411-415 (1995)).

About 17 million people, including 5 million children, in the United States have asthma, which amounts to 6.4% of the United States population, according to the National Institute of Allergy and Infectious Diseases. Other agencies provide similar estimates: 8.1 million children (National Health Interview Survey, 1997); 51 per 1000 (National Health Interview Survey, 1995); 14.5 million or 5% of the United States population (National Womens Health Information Center’); and 14.9 million in 1995 (National Heart, Lung, and Blood Institute). Of these, 10 to 20% are aspirin-intolerant individuals, for whom aspirin therapy in the prevention of cerebrovascular and cerebrovascular arterial thrombosis is contra-indicated (Jenkins, C. et al., BMJ 328:434 (2004)). Accordingly, there is a large unmet need for methods of preventing or treating adverse cerebrovascular and cardiovascular events in patients who are aspirin-sensitive. The present invention provides methods and compositions that meet this unmet need.

### BRIEF SUMMARY OF THE INVENTION

It has been surprisingly discovered that an anti-thrombotic action of TP modulators is mediated by endogenous platelet agents that are inhibited by aspirin, and notably, in part, by PGD₂, an agent whose effects in some other systems is blocked by TP modulators. Accordingly, the present invention provides methods and compositions useful in the treatment or prevention of cardiovascular disorders in individuals for whom therapy with a COX-1 enzyme inhibitor is not feasible due to either sensitivity, intolerance, or resistance to the inhibitor. In certain embodiments, the COX-1 inhibitor is aspirin or an NSAID.

In one embodiment, the invention provides methods of treating these individuals by administering a therapeutically effective amount of a thromboxane A₂ receptor (TP) modulator, alone or in a combination therapy with an ADP receptor modulator. In particular embodiment, the TP modulator is an antagonist of the platelet TP or a mixed inhibitor of thromboxane synthetase. The TP modulator may or may not be a mixed TP antagonist or TP inhibitor. In particular embodiments, the ADP modulator is an antagonist or inactivator of the platelet ADP receptor or a modulator of human CD39 (e.g., recombinant soluble ecto-ADPase/CD39).

In some embodiments, the intolerance is acute asthma brought on by contact with aspirin or another COX-1 inhibitor. In other embodiments, the sensitivity is due to gastrointestinal bleeding induced by the COX-1 inhibitor. In still another embodiments, the sensitivity is due to an adverse effect of the COX-1 inhibitor on the kidney or its function. In some embodiments, the individual is not otherwise in need for an effect exacerbated or induced by the administration of a COX-1 inhibitor. In some further embodiments, the individual is not concurrently in need for asthma therapy aside for asthma brought on by administration of a COX-1 inhibitor (e.g., aspirin).

In some embodiments, the individual is one known to be sensitive, intolerant, or resistant to a COX-1 inhibitor, or is predicted to be sensitive, intolerant, or resistant to a COX-1 inhibitor. In other embodiments, an individual is selected for the administration of the TP modulator by first querying the individual to determine whether they have had a prior adverse reaction following administration of aspirin or another NSAID in which an affirmative response identifies the individual as an aspirin-sensitive individual. In further embodiments, the adverse reaction is selected from the group consisting of: decreased forced expiratory volume, asthma, shortness of breath, difficulty breathing or swallowing, nausea, gastric bleeding, anemia or low blood cell count, rhinitus, nasal congestion, cough, urticaria, fainting, dizziness, or a drop in blood pressure.

In other embodiments, an individual is selected for the therapy by administering aspirin or NSAID to the individual and screening a sample from the individual for the presence of leukotriene E4 (LTE4), wherein the presence of an elevated LTE4 level in the sample identifies the individual as an aspirin sensitive individual. The sample can be, but is not limited to blood, plasma, serum or urine.

In still other embodiments, the individual is selected for therapy by administering a challenge dose of aspirin or other NSAID to an individual; and measuring the individual's forced expiratory volume (FEV₁), wherein a decreased FEV₁ identifies the individual as an aspirin sensitive individual.

In yet another embodiment, the aspirin sensitive individual is selected by administering aspirin or another NSAID to an individual; and measuring the individual's nasal volume, as by acoustic rhinometry wherein a decreased nasal volume identifies the individual as an aspirin sensitive individual. The aspirin may be administered intranasally or any other route of interest.

In other embodiments, the individual to be treated is a patient who has been found non-compliant with an aspirin or NSAID regimen for the treatment of any disorder, including, but not limited to, cardiovascular disorders, due to unwanted side effects.

In some embodiments of any of the above, the TP antagonist is ifetroban, 5-hexenoic acid, 6-[3-[[(cyanoamino)[(1,1-dimethylethyl)amino]methylene]amino]phenyl]-6-(3-pyridinyl)-, (epsilon)-) (terbogrel), 4-methoxy-N,N′-bis(3-pyridinylmethyl)-1,3-benzenedicarboxamide (picotamide), S-18886, 5-[(2-chlorophenyl)methyl]-4,5,6,7-tetrahydrothieno[3,2-c]pyridine, N-[2-(methylthio)ethyl]-2-[(3,3,3-trifluoropropyl)thio]-5′-adenylic acid, monoanhydride with dichloromethylenebisphosphonic acid, 2-(propylthio)-5′-adenylic acid, monoanhydride with dichloromethylene bis(phosphonic acid), methyl(+)-(S)-α-(2-chlorophenyl)-6,7-dihydrothieno[3,2-c]pyridine-5(4H)-acetate, 2-acetoxy-5-(α-cyclopropylcarbonyl-2-fluorobenzyl)-4,5,6,7-tetrahydrothieno[3,2-c]pyridine, and pharmaceutically acceptable salts thereof.

In some embodiments, the TP modulator or mixed TP antagonist has an nitric oxide (NO)-donating moiety. In some embodiments, the TP modulator or mixed TP agonist is administered with an NO donor or a compound which is metabolized to release NO in vivo. Such agents are well known in the art and include, but are not limited to, nitroglycerin and arginine.

In other embodiments of any of the above, the individual is further administered a HMG-CoA reductase inhibitor. These agents, commonly referred to as statins, include atorvastatin (Lipitor), simvastatin (Zocor), pravastatin (Pravachol), lovastatin (Mevacor), fluvastatin (Lescol), and rosuvastatin (Crestor). The reductase inhibitor may be administered separately or in combination with the TP modulator.

In some embodiments further of any of the above, the TP antagonist can be administered in a combination therapy with a direct thrombin inhibitor or a Factor Xa inhibitor. They may be administered separately or be co-formulated in single pharmaceutical composition.

In some embodiments of any of the above, the cardiovascular disorder is an acute coronary syndrome selected from the group consisting of: acute myocardial ischemia, acute myocardial infarction, and angina. In additional embodiments, the cardiovascular disorder is a thrombotic disorder selected from the group consisting of: atherosclerosis, thrombocytosis, peripheral artery occlusion, stenosis. In some embodiments, the aspirin-sensitive, intolerant, or resistant individual has a coronary stent. In one embodiment, the individual is schedule to undergo, is currently undergoing, or has recently undergone coronary artery bypass graft surgery.

In some embodiments of any of the above, the TP antagonist can be administered for as long as the benefit sought is desired. In particular embodiments, the TP antagonist is administered in a sustained release form in which the TP antagonist is administered biweekly, weekly, or monthly. A dosing period of at least one to two weeks may be required to initially achieve the benefit.

In particular embodiments, the ADP receptor antagonist or inactivator is a thienopyridine derivative (e.g., clopidogrel), prasugrel, or ticlopidine. In some further embodiments, the effective dose of the TP antagonist is reduced in the presence of the ADP receptor antagonist. The reduction may be by at least about 25%, 50%, or 75%.

In one embodiment, the ADP receptor antagonist has the structure shown in Formula I:

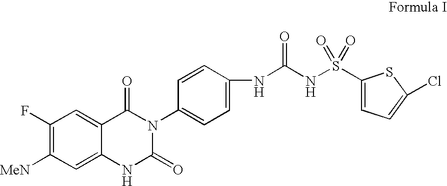

or is a pharmaceutically acceptable salt thereof.

In some of the embodiments of any of the above, PGD₂ levels in the individual are sufficient to mediate a dethrombotic action of the TP modulator. In some further embodiments, the invention provides a method of treatment by administering a thromboxane A2 receptor antagonist or a thromboxane synthase inhibitor and a second agent which inhibits or inactivates the ADP receptor (e.g., P2Y₁₂), wherein the treated individual PGD₂ levels are not affected by administration of a COX-1 inhibitor and thus can mediate a dethrombotic action of the first agent.

In another aspect, the invention relates to the surprising finding that PGD₂ is a potent dethrombotic agent that mediates, in part, the dethrombotic effect of TP modulators and that aspirin antagonizes this beneficial effect of TP modulators. In this aspect, the invention provides methods of treating cardiovascular disorders in a human individual subject who is receiving a therapeutically effective dose of a TP modulator and is instructed or advised to avoid and/or not to take aspirin or another COX-1 inhibitor. The instruction or advice may be in any media, including writing or delivered verbally. This advice can serve to maximize the dethrombotic activity of the TP modulator by avoiding the effect of aspirin or another COX-1 inhibitor on PGD₂ levels for example. In further embodiments, the subject is also being treated with a therapeutically effective dose of an ADP modulator. In some further embodiments of the above in this aspect, the individual to be treated is not aspirin sensitive, intolerant nor resistant or one for whom aspirin is otherwise contraindicated. In some further embodiments in this aspect, the individual has asthma that may or may not be aspirin sensitive. In some further embodiments in this aspect, the individual has coronary artery disease or an acute myocardial infarction or stroke. In other embodiments, the individual has an acute thrombotic event. In some embodiments, the individual is a subject having asthma and a cardiovascular disorder.

### DESCRIPTION OF THE DRAWINGS

FIG. 1 illustrates the downstream effects on COX-1 and COX 2 inhibition by aspirin on prostaglandin synthesis.

FIG. 2 illustrates the metabolic pathways affected by inhibition of COX-1 and how they relate to aspirin or COX-1 inhibitor sensitivity.

FIG. 3 illustrates the antithrombotic activity of ifetroban and aspirin in normal volunteers. The Y-axis represents fluorescence units, and the X-axis represents time in seconds.

FIG. 4 illustrates the antithrombotic profile of Ifetroban and aspirin in combination with clopidogrel. The Y-axis represents fluorescence units, and the X axis represents time in seconds.

FIG. 5 illustrates the effects of PGD₂ on thrombus stability. The Y-axis represents fluorescence units, and the X-axis time represents units in seconds. FIG. 5A illustrates dethrombus induced by a TP antagonist; FIG. 5B illustrates that continued TXA₂-induced signaling through TP is required to maintain stable thrombi; and FIG. 5C shows that the dethrombotic activity of a TP antagonist was prevented by prior aspirin treatment, and that addition of physiological concentrations of PGD₂ induced dethrombotic activity in aspirinated blood.

FIG. 6 sets forth some preferred TP modulators for use according to the invention.

FIG. 7 provides graphs depicting the antithrombotic activity of 100 nm or 350 nm ifetroban (103) versus aspirin in healthy volunteers (FIG. 7A) and aspirin-intolerant (AERD)-asthmatic patients (FIG. 7B) as determined using a perfusion chamber assay. The inhibition of thrombosis is shown as a reduction in the fluorescence intensity as compared to controls.

FIG. 8 provides bar graphs showing the antithrombotic activity of 1 μM, 100 nM, and 350 nM ifetroban (103) versus aspiring in healthy volunteers (FIG. 8A) and aspirin-intolerant (AERD)-asthmatic patients (FIG. 8B) as determined using a collagen-induced platelet aggregation assay. As shown, a statistically significant inhibition of collagen-induced platelet aggregation was shown at concentrations >100 nM in both normal volunteers (P=0.0281) and AERD patients. NS indicates not significant.

FIG. 9 is a graph showing the antithrombotic activity of 2 mM SQ29548 and 5 μM terbogrel as compared to aspirin in an aspirin-intolerant (AERD)-asthmatic patient, as determined using a perfusion chamber assay.

FIG. 10 provides graphs depicting the antithrombotic activity of 1 μM, 350 nM, and 100 nM ifetroban versus aspirin in healthy volunteers (FIG. 10A) and aspirin-intolerant (AERD-asthmatic patients (FIG. 10B) as determined by an arachidonic acid-induced platelet aggregation assay.

### DETAILED DESCRIPTION OF THE INVENTION

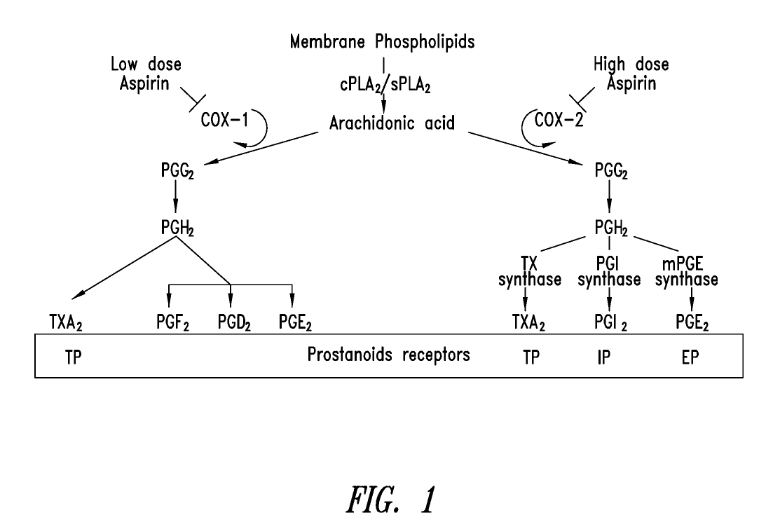

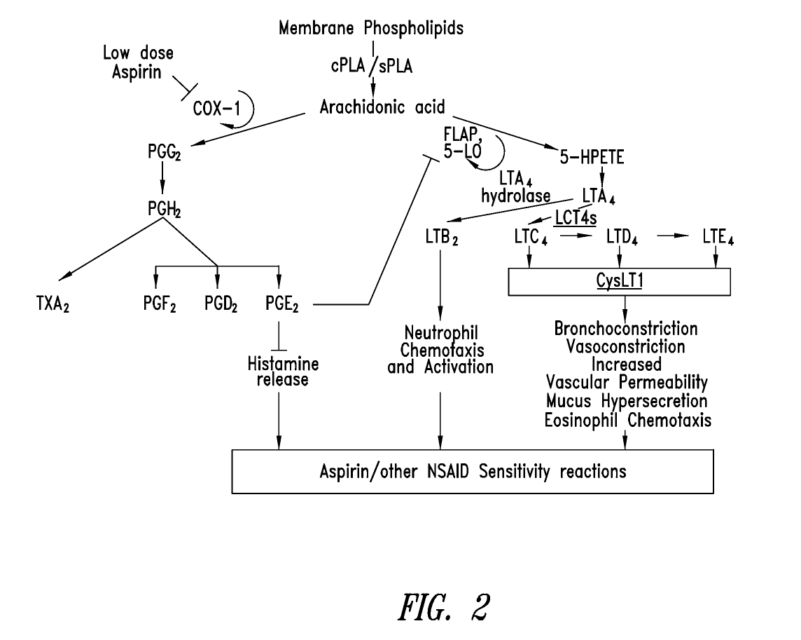

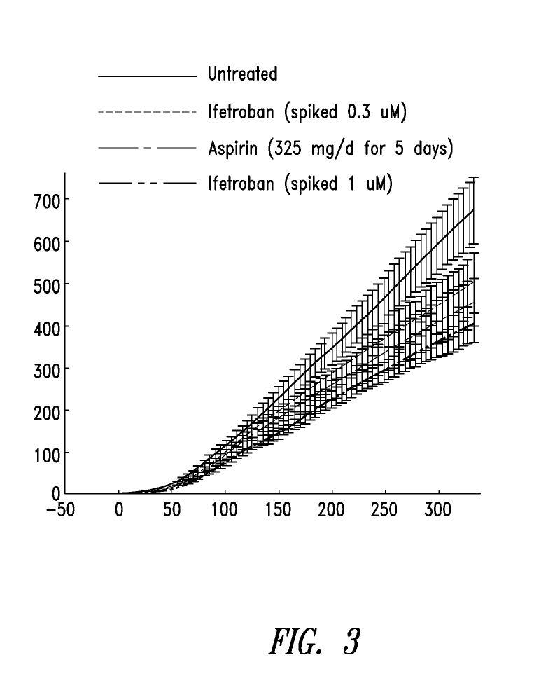

The present invention is based, in part, on the surprising discovery that an anti-thrombotic action of TP modulators is mediated by platelet endogenous agents whose synthesis is prevented by the use of aspirin, and, in part, by PGD₂, an agent whose effects in some other systems is blocked by TP modulators. The synthesis of PGD₂ is known to be inhibited by COX-1 inhibitors (see FIG. 1). Since TP antagonism does not inhibit PGE₂ (considered to be the main cause of the aspirin-intolerance reaction) and does not block PGD₂ production, the present invention establishes that targeting TP is an ideal aspirin-replacement therapy for use in aspirin-sensitive, aspirin-intolerant, and aspirin-resistance individuals.

Accordingly, the present invention provides methods and compositions useful in the treatment or prevention of diseases, disorders, and injuries, including, e.g., cardiovascular disorders, in individuals for whom therapy with a COX-1 enzyme inhibitor is not feasible due to sensitivity, intolerance, or resistance to the inhibitor. Additionally, the invention provides methods of treating diseases and disorders in an individual, comprising providing to the subject a therapeutically effective dose of a TP modulator and instructing or advising the individual to avoid and/or not to take aspirin or any other COX-1 inhibitor. The present invention further provides compositions and kits suitable for practicing the methods of the present invention.

### DEFINITIONS

In accordance with the invention and as used herein, the following terms are defined with the following meanings, unless explicitly stated otherwise.

The article “a” and “an” as used herein refers to one or to more than one (i.e., at least one) of the grammatical object of the article. By way of example, “an element” means one element or more than one element.

The term “aspirin” or “ASA” refers to ortho-acetylsalicylic acid and the pharmaceutically acceptable formulations thereof.

The term “non-steroidal anti-inflammatory drug” or “NSAID” refers to drugs with analgesic, antipyretic and anti-inflammatory activity and which are not steroids. The NSAIDs herein are limited to COX-1 inhibitors.

The term “aspirin-intolerant individual” or “COX-1 inhibitor intolerant individual” as used herein refers to an individual who has had, or is likely to have, an asthmatic response to aspirin (e.g., orally, intravenously, intratracheally, or intranasally) or another COX-1 inhibitor. One representation of the aspirin-induced or COX-1 inhibitor-induced asthma can be chronic, including individuals with eosinophilic inflammation of the upper and lower airways, most commonly including elevated baseline excretion of N-acetyl LTE4 as a marker of cysLT generation. COX-1 inhibitor induced asthma or aspirin-induced asthma also refers to the acute disease, where aspirin, or for example, NSAIDs, elicit life-threatening bronchospasm, which can be accompanied by mucocutaneous manifestations such as cutaneous hives and abdominal colic. The mucocutaneous form can occur without a background of bronchial asthma and is poorly related to atopy or eosinophilia. The term includes individuals with a history of bronchospasm or angioedema that has been associated with aspirin or non-steroidal anti-inflammatory drug (NSAID) use. The term also includes patients in whom ASA/NSAID use is contraindicated due to a history of bronchospasm, angioedema or nasal polyps that have occurred in conjunction with ASA/NSAID administration and that has been confirmed by either a positive provocative challenge test (nasal, oral or bronchial) or an elevated excretion of N-acetyl LTE4.

The term “aspirin-resistant” or “COX-1 inhibitor resistant” refers to individuals who have a demonstrated lack of effect of ASA and/or NSAIDs or other COX-1 enzyme inhibitors on bleeding time and/or platelet function.

The term “aspirin-sensitive” or “COX-1 inhibitor-sensitive” refers to patients who are unable to take ASA/NSAIDs due to aspirin intolerance (see above), a history of active GI disease (such as gastric ulcers), GI symptoms linked to ASA/NSAID use (such as heartburn, nausea or abdominal pain), or a bleeding diathesis.

The term “cardiovascular disorder” as used herein refers to any disease or disorder affecting the heart and circulatory system, including thrombotic disorders (i.e., disorders involving formation of a clot in a blood vessel; a clot can be made of platelets, red blood cells, fibrin, leukocytes) and acute coronary syndrome (e.g., acute myocardial ischemia, acute myocardial infarction, and stable or unstable angina) and cerebrovascular disorders (e.g., stroke associated with thrombosis), myocardial infarction, stable or unstable angina, reocclusion after PTCA, restenosis after PTCA, as well as intermittent claudication, transient ischemic attacks, stroke, e.g., ischemic stroke, and reversible ischemia neurological deficit. Methods of diagnosing patients with the subject cardiovascular disorders are known to one of ordinary skill in the medical arts.

The terms “thromboxane” or “TX” as used herein refer to a member of the family of lipids known as eicosanoids. Thromboxane is produced in platelets by thromboxane synthetase and act to promote vasoconstriction, platelet aggregation, and bronchoconstriction in the lung. Thromboxane is an important mediator of vessel constriction, platelet aggregation and platelet adhesiveness. Thromboxane A₂ or TXA₂ refer to the active form of thromboxane and thromboxane B₂ or TXB₂ refer to the inactive form of thromboxane.

The terms “thromboxane receptor” or “TP” as used herein refer to the cellular receptor for thromboxane. TP is expressed on a number of different cell types including, e.g., smooth muscle cells, endothelial cells and platelets. Nucleic acid sequences encoding thromboxane receptors are set forth in Genbank Accession Nos. NM_{—}001060; NM_{—}201636; U30503; and E03829.

COX-1 inhibitor NSAIDs include, but are not limited to, aspirin, indobufen, flurbiprofen, naproxen, oxaprozin, indomethacin, ketorolac, mefenamic acid, nabumetone, ibuprofen, acetaminophen, etodolac. A preferred COX-1 inhibitor is aspirin. Therapeutic effects of the TP modulators, TP antagonists, thromboxane synthase inhibitors, and ADP receptor modulators for use according to the invention are not mediated through inhibition of Cox-1.

It will be understood that, unless otherwise indicated, reference to an ADP receptor modulator or thromboxane A2 receptor modulator includes all pharmaceutically acceptable forms of the drug known in the art. For example, any pharmaceutically acceptable salt of a drug may be used in compositions. The dosages recited however, refer to the drug itself, for example in its acid or base form.

When used with respect to a health condition, the term “chronic” indicates means a long-lasting condition which may be life-long or persist of indefinite length, usually lasting for one or more months. When used with respect to the administration of a pharmaceutical agent, the terms “chronic,” “chronically” and the like refer to an administration which usually lasting for a period of at least one or more months and which may be for an indefinite period.

As used herein, unless the context makes clear otherwise, “treat,” and similar word such as “treatment,” “treating” etc., is an approach for obtaining beneficial or desired results, including and preferably clinical results. Treatment can involve optionally either the reducing or amelioration of a disease or condition, (e.g., thrombosis or a related disease or disorder), or the delaying of the progression of the disease or condition.

As used herein, unless the context makes clear otherwise, “prevent,” and similar word such as “prevention,” “preventing” etc., is an approach for preventing the onset or recurrence of a disease or condition, (e.g., thrombosis or a related disease or disorder) or preventing the occurrence or recurrence of the symptoms of a disease or condition, or optionally an approach for delaying the onset or recurrence of a disease or condition or delaying the occurrence or recurrence of the symptoms of a disease or condition.

Generally, a subject is provided with an effective amount of an antithrombotic agent or an effective amount is used for its intended purpose. As used herein, an “effective amount” or a “therapeutically effective amount” of a substance, e.g., an antithrombotic agent, is that amount sufficient to affect a desired biological or psychological effect, such as beneficial results, including clinical results. For example, in the context of certain embodiments of the methods of the present invention, an effective amount of an antithrombotic agent is that amount sufficient to reduce or ameliorate thrombosis in vivo or ex vivo, or a related disease or disorder.

Methods of diagnosing patients with the subject diseases and disorders are known to one of ordinary skill in the art.

### A. Methods of Treating and Preventing Thrombosis and Other Diseases and Disorders

Methods of the present invention may be practiced both in vitro and in vivo to inhibit, reduce, or prevent platelet aggregation or blood coagulation, or to treat or prevent thrombosis and other diseases and disorders. In one embodiment, methods of the present invention are practiced on platelet preparations being stored prior to use. In other embodiments, methods of the present invention are practiced in vivo on individuals, e.g., patients, which include mammals and, in particular, humans.

In certain embodiments, methods of the present invention comprise providing one or more TP antagonists, alone or in combination with one or more additional therapeutic agents, to a subject having been determined to be aspirin-resistant, aspirin-sensitive, or aspirin-intolerant. In addition, in other embodiments, methods of the present invention comprise contacting a platelet with one or more TP antagonists, alone or in combination with on or more additional therapeutic agents. The platelets may be present within a subject, or they may be removed from a subject, permanently or temporarily.

The methods of the present invention may be practiced using an effective amount of a TP modulator, e.g., a TP antagonist, alone or in combination with one or more additional therapeutic agents. In particular embodiments, an additional therapeutic agent is an antithrombotic agent. In certain embodiments, an additional therapeutic agent is an ADP modulator, e.g., an ADP receptor antagonist or a CD39 modulator, or an HMGCoA reductase inhibitor. When used in combination with one or more additional therapeutic agents, the TP modulator and the one or more additional therapeutic agents may be provided to the subject or platelet at the same time or at different times, and by the same or different routes of administration.

In various embodiments, methods of the present invention are practiced to treat or prevent any disease or disorder treated or prevented by aspirin or another COX-1 inhibitor, or an NSAID. In addition, the methods of the present invention may be practiced during medical procedures, e.g., to prevent platelet aggregation or injury to an individual. Examples of particular diseases and disorders, as well as medical procedures, that may benefit from the methods of the present invention are described below.

Arterial thrombosis and disorders of coagulation are associated with a variety of cardiovascular-related diseases and disorders, including but not limited to, myocardial infarction, e.g., acute myocardial infarction, thrombotic stroke, atherosclerotic disease, unstable angina, refractory angina, transient ischemic attacks, embolic stroke, disseminated intravascular coagulation, septic shock, deep venous thrombosis, pulmonary embolism, reocclusion, restenosis, pulmonary embolism, and occlusive coronary thrombus or other complications resulting from thrombolytic therapy, percutaneous transluminal coronary angioplasty, or coronary artery bypass grafts (CABG). In addition, the methods and compositions of the present invention may be used to treat or prevent pulmonary hypertension, e.g., hypoxia-induced pulmonary hypertension, and intravascular thrombosis, which have been linked to Cox-2 (Cathcart, M. C. et al., J. Pharmacol. Exp. Ther. Mar. 28, 2008 DOI: 10.1124/jpet.107.134221).

Aspirin is frequently prescribed for patients who are having a heart attack to limit the extent of damage to the heart muscle, prevent additional heart attacks, and improve survival. Aspirin is also often prescribed on a long-term basis to patients with prior heart attacks or stroked and to patients with transient ischemic attack (TIA) and exertional angina to prevent heart attacks and ischemic strokes. Aspiring is also prescribed to patients having unstable angina to prevent heart attacks and improve survival. In addition, aspirin is prescribed to patients who are having ischemic strokes to limit damage to the brain, prevent another stroke, and improve survival.

Methods of the present invention may be used in the treatment or prevention of any of these and other thrombosis or coagulation-related diseases and disorders. The TP modulators with, optionally, ADP receptor modulators are especially useful in the treatment and prevention of peripheral arterial disease, arterial or venous thrombosis, unstable angina, transient ischemic attacks and hypertension in COX-1 inhibitor -sensitive, -intolerant, or -resistant individuals. Thus, the inventive therapy avoids the use of COX-1 inhibitors, including particularly aspirin.

In particular embodiments, the present invention includes a method of inhibiting, reducing, or preventing platelet aggregation or blood coagulation, comprising providing a TP antagonist to an aspirin-resistant, aspirin-sensitive or aspirin-intolerant patient at risk of or diagnosed with platelet aggregation or blood coagulation.

In related embodiments, the present invention includes a method of treating or preventing thrombosis or a cardiovascular diseases or disorder comprising providing a TP antagonist to an aspirin-resistant, aspirin-sensitive or aspirin-intolerant patient at risk of or diagnosed with platelet aggregation or blood coagulation.

In another related embodiment, the present invention includes a method of treating of preventing thrombosis or a thrombotic event in a patient during coronary artery bypass surgery, e.g., CABG, comprising providing a TP antagonist to an aspirin-resistant, aspirin-sensitive or aspirin-intolerant patient undergoing or scheduled to undergo a coronary artery bypass surgery. Aspirin is often prescribed to patients undergoing surgery to open or bypass blocked arteries, including percutaneous transluminal coronary angioplasty (PTCA) with or without placement of coronary stents and coronary artery bypass surgery (CABG). Aspirin is also prescribed on a long-term basis to prevent clotting in the stents and/or the bypassed blood vessels.

In various embodiments of the present invention, the TP modulator and, optionally, the ADP receptor modulator, are provided to the individual during a medical procedure or prior to a medical procedure. Generally, the TP modulator and, optionally, the ADP receptor modulator, are provided to the individual in amounts effective to reduce platelet aggregation or thrombosis during or after the medical procedure, e.g., coronary surgery.

Cardiopulmonary surgery may be performed using cardiopulmonary bypass, e.g., on-pump coronary artery bypass surgery. However, this is frequently associated with a decrease in platelet count and an increase in platelet activation. The present invention provides methods for performing on-pump coronary bypass surgery comprising providing a TP antagonist, alone or in combination with an ADP receptor antagonist, prior to or during on-pump coronary bypass surgery. This method should reduce platelet loss during the procedure.

The methods of the present invention are also advantageous for the treatment of patients undergoing dialysis. In particular, a patient may be provided with a TP antagonist, alone or in combination with an ADP receptor antagonist, prior to or during dialysis, in order to prevent clotting and reduce loss of platelets, which may adhere or clump in the dialysis pump unit. Alternatively, or in addition, the pump unit or a portion thereof that comes into contact with the patient's blood, such as a tube, may be coated with a TP antagonist, alone or in combination with an ADP receptor antagonist.

Similarly, a stent or other device may be coated with a TP antagonist, alone or in combination with an ADP receptor antagonist, in order to prevent clotting in the stent or near the area of the stent.

Thrombocythemia or thrombocytosis is characterized by an increased production of platelets in the bone marrow, which can lead to increased blood clotting. Accordingly, the present invention provides a method of treating thrombocythemia or thrombocytosis by providing to a patient, e.g., an aspirin-sensitive or aspirin-resistance patient, diagnosed with or at risk of this disease a TP antagonist, optionally in combination with an ADP receptor antagonist.

Methods of the present invention may also be adapted for the treatment and prevention of acute lung injury, e.g., in aspirin-sensitive or aspirin-resistant individuals. Acute lung injury or hypoxemic respiratory failure, a severe version of which is acute respiratory distress syndrome (ARDS) is frequently associated with a systemic inflammatory process, such as sepsis, and is also caused by trauma, pneumonia, and burns, etc. Patients with acute lung injury are typically ventilated, and aspirin treatment is contraindicated. The present invention provides a method of treating acute lung injury comprising providing a TP antagonist, alone or in combination with an ADP receptor antagonist, to an individual having an acute lung injury.

Methods of the present invention, which provide a substitute to treatment with aspirin or COX-1 inhibitors are also advantageous in the treatment of children having or recovering from a viral infection, who are at risk of developing Reyes Syndrome from aspirin. Accordingly, the present invention provides a method for treating an individual at risk of developing Reyes syndrome in response to aspirin or other salicylate, comprising providing to the individual a TP antagonist, alone or in combination with an ADP receptor antagonist. In certain embodiments, the methods are practiced to reduce pain, discomfort, or fever.

In certain embodiments, the methods further comprises determining whether a patient is aspirin-sensitive or aspirin-intolerant, e.g., by questioning the patient. In particular embodiments of the methods of the present invention, patients are also instructed to not take aspirin or any another COX-1 inhibitor.

In other embodiments, any of the methods further includes providing an ADP modulator, e.g., an ADP receptor antagonist, to the patient. The TP modulator and ADP modulator may be provided to the patient at the same time or in any order.

The TP modulators and, optionally, ADP modulators, should be given in a co-timely manner and should be delivered in an amount sufficient to render the desired benefit. Preferably, the modulators are delivered together in a unit dosage form as described herein. The duration of therapy will be in accordance with the duration of the disorder to be treated. Typically, therapy will be chronic given the chronic nature of many of the recited cardiovascular conditions or the need for chronic prevention.

In particular embodiments, methods of the present invention are practiced using higher dosages or higher blood plasma concentrations of a TP modulator than previously thought necessary or previously used to treat patients. These may be, e.g., at least two-fold, at least three-fold, at least four-fold, at least five-fold, at least six-fold, at least seven-fold, at least eight-fold, at least nine-fold, or at least ten-fold higher than concentrations determined to be effective at inhibiting platelet aggregation using a standard in vitro U-46619-induced platelet aggregation assay.

The amount of TP modulator, and optionally, ADP modulator, may be administered as a single dose, or in may be administered periodically to maintain the desired blood plasma concentration. For example, it may be administered every 6, 12, 24, 48, or 72 hours.

In particular embodiments, a TP modulator is administered in an amount sufficient to achieve a blood plasma concentration greater than or equal to 1, 5, 10, 20, 50, 100, 200, 300, 400, 500, 600, 700, 800, 900, or 1000 nM. In particular embodiments, it is administered in an amount sufficient to achieve a blood plasma level greater than or equal to 350 nM. In certain embodiments, it is administered in an amount sufficient to achieve a blood plasma concentration in the range of 1-10 nM, 1-100 nM, 10-1000 nM, 50-500 nM, 100-500 nM, 200-400 nM, 200-1000 nM, or 500-1000 nM.

In particular embodiments, ifetroban or other TP modulator is administered in an amount sufficient to achieve a blood plasma concentration of at least 100 nM, at least 150 nM, at least 200 nM, at least 250 nM, at least 300 nM, or at least 350 nM. In certain embodiments, ifetroban is administered in an amount sufficient to maintain a blood concentration of at least 100 nM, at least 150 nM, at least 200 nM, at least 250 nM, at least 300 nM, or at least 350 nM for at least 6, 12, 24, or 48 hours.

In particular embodiments, ifetroban or other TP modulator is administered in an amount within the range of from about 0.01 mg/kg to about 100 mg/kg, from about 0.1 mg/kg to about 100 mg/kg, from about 1 mg/kg to about 100 mg/kg, or from about 10 mg/kg to about 100 mg/kg. In particular embodiments, an antithrombotic agent is administered in an amount within the range of about 1 mg/kg to about 10 mg/kg, from about 2 mg/kg to about 10 mg/kg, from about 4 mg/kg to about 8 mg/kg or about 6 mg/kg to about 8 mg/kg. In one embodiment, it is administered at approximately 7 mg/kg.

Other compounds, such as ADP modulators, may be administered at therapeutically effective amounts, including any of the dosages and ranges described herein. The amount of composition administered will, of course, be dependent on the subject being treated, on the subject's weight, the severity of the affliction, the manner of administration and the judgment of the prescribing physician. Determination of an effective amount is well within the capability of those skilled in the art, especially in light of the detailed disclosure provided herein.

1. TP Modulators

The term “thromboxane A2 receptor antagonist” or “thromboxane receptor antagonist” or “TP antagonist” as used herein refers to a compound that inhibits the expression or activity of a thromboxane receptor by at least or at least about 30%, 35%, 40%, 45%, 50%, 55%, 60%, 65%, 70%, 75%, 80%, 85%, 90%, 95%, 96%, 97%, 98%, 99%, or 100% in a standard bioassay or in vivo or when used in a therapeutically effective dose. In certain embodiments, a TP antagonist inhibits binding of thromboxane A₂ to TP. TP antagonists include competitive antagonists (i.e., antagonists that compete with an agonist for TP) and non-competitive antagonists. TP antagonists include antibodies to the receptor. The antibodies may be monoclonal. They may be human or humanized antibodies. TP antagoninsts also include thromboxane synthase inhibitors, as well as compounds that have both TP antagonist activity and thromboxane synthase inhibitor activity.

TP antagonists include, for example, small molecules such as ifetroban (BMS; [1S-(1α,2α,3α,4α)]-2-[[3-[4-[(pentylamino)carbonyl]-2-oxazolyl]-7-oxabicyclo[2.2.1]hept-2-yl]methyl]benzenepropanoic acid), 5-hexenoic acid, 6-[3-[[(cyanoamino)[(1,1-dimethylethyl)amino]methylene]amino]phenyl]-6-(3-pyridinyl)-, (E-) (terbogrel), 5-[(2-chlorophenyl)methyl]-4,5,6,7-tetrahydrothieno[3,2-c]pyridine, N-[2-(methylthio)ethyl]-2-[(3,3,3-trifluoropropyl)thio]-5′-adenylic acid, monoanhydride with dichloromethylenebisphosphonic acid, 2-(propylthio)-5′-adenylic acid, monoanhydride with dichloromethylene bis(phosphonic acid), methyl(+)-(S)-α-(2-chlorophenyl)-6,7-dihydrothieno[3,2-c]pyridine-5(4H)-acetate, 2-acetoxy-5-α-cyclopropylcarbonyl-2-fluorobenzyl)-4,5,6,7-tetrahydrothieno[3,2-c]pyridine, 4-methoxy-N,N′-bis(3-pyridinylmethyl)-1,3-benzenedicarboxamide (picotamide), ridogrel (Janssen), sulotroban, UK-147535 (Pfizer), GR 32191 (Glaxo), variprost, and S-18886 (Servier).

Additional TP antagonists suitable for use herein are also described in U.S. Pat. No. 6,509,348. These include, but are not limited to, the interphenylene 7-oxabicycloheptyl substituted heterocyclic amide prostaglandin analogs as disclosed in U.S. Pat. No. 5,100,889, issued Mar. 31, 1992, including [1S-(1α,2α,3α,4α)]-2-[[3-[4-[[(4-cyclohexylbutyl)amino]carbonyl]-2-oxazolyl]-7-oxabicyclo[2.2.1]hept-2-yl]methyl]benzenepropanoic acid (SQ 33,961) which is preferred, or esters or salts thereof; [1S-(1α,2α,3α,4α)]-2-[[3-[4-[[[(4-chlorophenyl)butyl]amino]carbonyl]-2-oxazolyl]-7-oxabicyclo[2.2.1]hept-2-yl]methyl]benzenepropanoic acid or esters, or salts thereof; [1S-((1α,2α,3α,4α)]-3-[[3-[4-[[(4-cyclohexylbutyl)amino]carbonyl]-2-oxazolyl]-7-oxabicyclo[2.2.1]hept-2-yl]methyl]benzeneacetic acid, or esters or salts thereof; [1S-((1α,2α,3α,4α)]-[2-[[3-[4-[[(4-cyclohexylbutyl)amino]carbonyl]-2-oxazolyl]-7-oxabicyclo[2.2.1]hept-2-yl]methyl]phenoxy]acetic acid, or esters or salts thereof; [1S-((1α,2α,3α,4α)]-2-[[3-[4-[[-7,7-dimethyloctyl)amino]carbonyl]-2-oxazolyl]-7-oxabicyclo[2.2.1]hept-2-yl]methyl]benzenepropanoic acid, or esters or salts thereof and ifetroban; 7-oxabicycloheptyl substituted heterocyclic amide prostaglandin analogs as disclosed in U.S. Pat. No. 5,100,889, issued Mar. 31, 1992, including [1S-[1α,2α(Z),3α,4α)]-6-[3-[4-[[(4-cyclohexylbutyl)amino]carbonyl]-2-oxazolyl]-7-oxabicyclo[2.2.1]hept-2-yl]-4-hexenoic acid, or esters or salts thereof; [1S-[1α,2α(Z),3α,4α)]]-6-[3-[4-[[(4-cyclohexylbutyl)amino]carbonyl]-2-thiazolyl]-7-oxabicyclo[2.2.1]hept-2-yl]-4-hexenoic acid, or esters or salts thereof; [1S-[1α,2α(Z),3α,4α)]]-6-[3-[4-[[(4-cyclohexylbutyl)methylamino]carbonyl]-2-oxazolyl]-7-oxabicyclo[2.2.1]hept-2-yl]-4-hexenoic acid, or esters or salts thereof; [1S-[1α,2α(Z),3α,4α)]]-6-[3-[4-[(1-pyrrolidinyl)carbonyl]-2-oxazolyl]-7-oxabicyclo[2.2.1]hept-2-yl]-4-hexenoic acid, or esters or salts thereof; [1S-[1α,2α(Z),3α,4α)]]-6-[3-[4-[(cyclohexylamino)carbonyl]-2-oxazolyl]-7-oxabicyclo[2.2.1]hept-2-yl-4-hexenoic acid or esters or salts thereof; [1S-[1α,2α(Z),3α,4α)]]-6-[3-[4-[[(2-cyclohexylethyl)amino]carbonyl]-2-oxazolyl]-7-oxabicyclo[2.2.1]hept-2-yl]-4-hexenoic acid, or esters or salts thereof; [1S-[1α,2α(Z),3α,4α)]]-6-[3-[4-[[[2-(4-chloro-phenyl)ethyl]amino]carbonyl]-2-oxazolyl]-7-oxabicyclo-[2.2.1]hept-2-yl]-4-hexenoic acid, or esters or salts thereof; [1S-[1α,2α(Z),3α,4α)]]-6-[3-[4-[[(4-chlorophenyl)amino]carbonyl]-2-oxazolyl]-7-oxabicyclo[2.2.1]hept-2-yl]-4-hexenoic acid, or esters or salts thereof; [1S-[1α,2α(Z),3α,4α)]]-6-[3-[4-[[[4-(4-chlorophenyl)butyl]amino]carbonyl]-2-oxazolyl]-7-oxabicyclo[2.2.1]hept-2-yl]-4-hexenoic acid, or esters or salts thereof; [1S-[1α,2α(Z),3α,4α)]]-6-[3-[4α-[[(6-cyclohexylhexyl)amino]carbonyl]-2-oxazolyl]-7-oxabicyclo[2.2.1]hept-2-yl]-4-hexenoic acid, or esters, or salts thereof; [1S-[1α,2α(Z),3α,4α)]]-6-[3-[4-[[(6-cyclohexylhexyl)amino]carbonyl]-2-oxazolyl]-7-oxabicyclo[2.2.1]hept-2-yl]-4-hexenoic acid, or esters or salts thereof; [1S-[1α,2α(Z),3α,4α)]]-6-[3-[4-[(propylamino)carbon yl]-2-oxazolyl]-7-oxabicyclo[2.2.1]hept-2-yl]-4-hexenoic acid, or esters or salts thereof; [1S-[1α,2α(Z),3α,4α)]]-6-[3-[4-[[(4-butylphenyl)amino]carbonyl]-2-oxazolyl]-7-oxabicyclo[2.2.1]hept-2-yl]-4-hexenoic acid, or esters or salts thereof; [1S-[1α,2α(Z),3α,4α)]]-6-[3-[4-[(2,3-dihydro-1H-indol-1-yl)carbonyl]-2-oxazolyl]-7-oxabicyclo[2.2.1]hept-2-yl]-4-hexenoic acid, or esters or salts thereof; [1S-[1α,2α(Z),3α,4α)]]-6-[3-[4-[[(4-cyclohexylbutyl)amino]carbonyl]-2-oxazolyl]-7-oxabicyclo[2.2.1]hept-2-yl]-N-(phenylsulfonyl)-4-hexenamide; [1S-[1α,2α(Z),3α,4α)]]-6-[3-[4-[[(4-cyclohexylbutyl)amino]carbonyl]-2-oxazolyl]-N-(methylsulfonyl)-7-oxabicyclo[2.2.1]hept-2-yl]-4-hexenamide; [1S-[1α,2α(Z),3α,4α)]]-7-[3-[4-[[(4-cyclohexylbutyl)amino]carbonyl]-2-oxazolyl]-7-oxabicyclo[2.2.1]hept-2-yl]-5-heptenoic acid, or esters or salts thereof; [1S-[1α,2α(Z),3α,4α)]]-6-[3-[4-[[(4-cyclohexylbutyl)amino]carbonyl]-1H-imidazol-2-yl]-7-oxabicyclo-[2.2.1]hept-2-yl]-4-hexenoic acid or esters or salts thereof; [1S-[1α,2α,3α,4α)]-6-[3-[4-[[(7,7-dimethyloctyl)amino]carbonyl]-2-oxazolyl]-7-oxabicyclo[2.2.1]hept-2-yl]-4-hexenoic acid, or esters or salts thereof; [1S-[1α,2α(E),3α,4α)]]-6-[3-[4-[[(4-cyclohexylbutyl)amino]carbonyl]-2-oxazolyl]-7-oxabicyclo[2.2.1]hept-2-yl]-4-hexenoic acid; [1S-[1α,2α,3α,4α)]-3-[4-[[(4-(cyclohexylbutyl)amino]carbonyl]-2-oxazolyl]-7-oxabicyclo[2.2.1]heptane-2-hexanoic acid or esters or salts thereof, with a preferred compound being [1S-[1α,2α(Z),3α,4α)]]-6-[3-[4-[[(4-cyclohexylbutyl)amino]carbonyl]-2-oxazolyl]-7-oxabicyclo-[2.2.1]hept-2-yl]-4-hexenoic acid, or esters or salts thereof; 7-oxabicycloheptane and 7-oxabicycloheptene compounds disclosed in U.S. Pat. No. 4,537,981 to Snitman et al., especially [1S-[1α,2α(Z),3α(1E,3S*,4R*),4α)]]-7-[3-(3-hydroxy-4-phenyl-1-pentenyl)-7-oxabicyclo[2.2.1]hept-2-yl]-5-heptenoic acid (SQ 29,548); the 7-oxabicycloheptane substituted amino prostaglandin analogs disclosed in U.S. Pat. No. 4,416,896 to Nakane et al., especially, [1S-[1α,2α(Z),3α,4α)]]-7-[3-[[2-(phenylamino)carbonyl]hydrazino]methyl]-7-oxabicyclo[2.2.1]hept-2-yl]-5-heptenoic acid; the 7-oxabicycloheptane substituted diamide prostaglandin analogs disclosed in U.S. Pat. No. 4,663,336 to Nakane et al., especially, [1S-[1α,2α(Z),3α,4α)]]-7-[3-[[[[(1-oxoheptyl)amino]acetyl]amino]methyl]-7-oxabicyclo[2.2.1]hept-2-yl]-5-heptenoic acid and the corresponding tetrazole, and [1S-[1α,2α(Z),3α,4α)]]-7-[3-[[[[(4-cyclohexyl-1-oxobutyl)amino]acetyl]amino]methyl]-7-oxabicyclo]2.2.1]hept-2-yl]-5-heptenoic acid; 7-oxabicycloheptane imidazole prostaglandin analogs as disclosed in U.S. Pat. No. 4,977,174, issued Dec. 11, 1990, including [1S-[1α,2α(Z),3α,4α)]]-6-[3-[[4-(4-cyclohexyl-1-hydroxybutyl)-1H-imidazole-1-yl]methyl]-7-oxabicyclo[2.2.1]hept-2-yl]-4-hexenoic acid or its methyl ester; [1S-[1α,2α(Z),3α,4α)]]-6-[3-[[4-(3-cyclohexylpropyl)-1H-imidazol-1-yl]methyl]-7-oxabicyclo[2.2.1]hept-2-yl]-4-hexenoic acid or its methyl ester; [1S-[1α,2α(Z),3α,4α)]]-6-[3-[[4-(4-cyclohexyl-1-oxobutyl)-1H-imidazol-1-yl]methyl]-7-oxabicyclo[2.2.1]hept-2-yl]-4-hexenoic acid or its methyl ester; [1S-[1α,2α(Z),3α,4α)]]-6-[3-(1H-imidazol-1-ylmethyl)-7-oxabicyclo[2.2.1]hept-2-yl]-4-hexenoic acid or its methyl ester; or [1S-[1α,2αZ),3α,4α)]]-6-[3-[[4-[[(4-cyclohexylbutyl)amino]carbonyl]-1H-imidazol-1-yl]methyl-7-oxabicyclo-[2.2.1]hept-2-yl]-4-hexenoic acid, or its methyl ester; the phenoxyalkyl carboxylic acids disclosed in U.S. Pat. No. 4,258,058 to Witte et al., especially 4-[2-(benzenesulfamido)ethyl]phenoxyacetic acid (BM 13, 177-Boehringer Mannheim), the sulphonamidophenyl carboxylic acids disclosed in U.S. Pat. No. 4,443,477 to Witte et al., especially 4-[2-(4-chlorobenzenesulfonamido)ethyl]phenylacetic acid (BM 13,505, Boehringer Mannheim), the arylthioalkylphenyl carboxylic acids disclosed in U.S. Pat. No. 4,752,616, especially 4-(3-((4-chlorophenyl)sulfonyl)propyl)benzeneacetic acid. yapiprost, (E)-5-[[[(pyridinyl)[3-(trifluoromethyl)phenyl]methylene]amino]oxy]pentanoic acid also referred to as R68,070-Janssen Research Laboratories, 3-[1-(4-chlorophenylmethyl)-5-fluoro-3-methylindol-2-yl]-2,2-dimethylpropanoic acid [(L-655240 Merck-Frosst) Eur. J. Pharmacol. 135(2):193, Mar. 17, 1987], 5(Z)-7-([2,4,5-cis]-4-(2-hydroxyphenyl)-2-trifluoromethyl-1,3-dioxan-5-yl) heptenoic acid (ICI 185282, Brit. J. Pharmacol. 90 (Proc. Suppl):228 P-Abs, March 87), 5(Z)-7-[2,2-dimethyl-4-phenyl-1,3-dioxan-cis-5-yl]heptenoic acid (ICI 159995, Brit. J. Pharmacol. 86 (Proc. Suppl):808 P-Abs., December 85), N,N′-bis[7-(3-chlorobenzeneaminosulfonyl)-1,2,3,4-tetrahydro-isoquinolyl]d isulfonylimide (SKF 88046, Pharmacologist 25(3):116 Abs., 117 Abs, August 83), [1α(Z)-2β,5α]-(+)-7-[5-[[(1,1′-biphenyl)-4-yl]methoxy]-2-(4-morpholinyl)-3-oxocyclopentyl]-4-heptenoic acid (AH 23848-Glaxo, Circulation 72(6):1208, December 85, levallorphan allyl bromide (CM 32,191 Sanofi, Life Sci. 31 (20-21):2261, Nov. 15, 1982), (Z,2-endo-3-oxo)-7-(3-acetyl-2-bicyclo[2.2.1]heptyl-5-hepta-3Z-enoic acid, 4-phenyl-thiosemicarbazone (EP092-Univ. Edinburgh, Brit. J. Pharmacol. 84(3):595, March 85); GR 32,191 (Vapiprost)-[1R-[1α(Z),2β,3β,5α]]-(+)-7-[5-([1,1′-biphenyl]-4-ylmethoxy)-3-hydroxy-2-(1-piperidinyl)cyclopentyl]-4-heptenoic acid; ICI 192,605-4(Z)-6-[(2,4,5-cis)-2-(2-chlorophenyl)-4-(2-hydroxyphenyl)-1,3-dioxan-5-yl]hexenoic acid; BAY u 3405 (ramatroban)-3-[[(4-fluorophenyl)sulfonyl]amino]-1,2,3,4-tetrahydro-9H-carbazole-9-propanoic acid; or ONO 3708-7-[2α,4α-(-(di-methylmethano)-6β-(2-cyclopentyl-2β-hydroxyacetamido)-1α-cyclohexyl]-5(Z)-heptenoic acid; (±)(5Z)-7-[3-endo-[(phenylsulfonyl)amino]bicyclo[2.2.1]hept-2-exo-yl]-heptenoic acid (S-1452, Shionogi domitroban, Anboxan™); (−)6,8-difluoro-9-p-methylsulfonylbenzyl-1,2,3,4-tetrahydrocarbazol-1-yl-acetic acid (L670596, Merck) and (3-[1-(4-chlorobenzyl)-5-fluoro-3-methyl-indol-2-yl]-2,2-dimethylpropanoic acid (L655240, Merck). TP antagonists that may be used according to the present invention also include benzenealkonic acids and benzenesulfonamide derivatives, typically at 1-1000 mg per unit dose and 1-5000 mg per day.

In one particular embodiment, the TP modulator is ifetroban, which is described above or alternatively described as: 3-[2-[[(1S,4R,5S,6R)-5-[4-(pentylcarbamoyl)-1,3-oxazol-2-yl]-7-oxabicyclo[2.2.1]hept-6-yl]methyl]phenyl]propanoate, or ifetroban sodium, which is sodium 3-[2-[[(1S,4R,5S,6R)-5-[4-(pentylcarbamoyl)-1,3-oxazol-2-yl]-7-oxabicyclo[2.2.1]hept-6-yl]methyl]phenyl]propanoate. As used herein, the term ifetroban includes both ifetroban and ifetroban sodium. The structure of ifetroban is shown in Formula I:

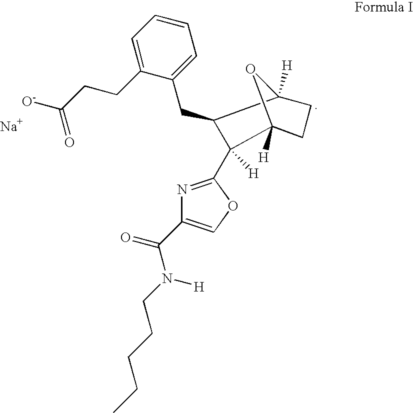

Certain exemplary TP modulators (e.g., Terbogrel (Boehringer Ingeliheim), Ifetroban, UK-147,535 (Pfizer), S 18886 (Servier), Seratodast-AA-2414 (Takeda/AstraZenica), Ramatroban (Bayer AG) and Ridogrel (Janssen), and BMI-531 (Univ. Liege) for use according to the invention are set forth in FIG. 3. In addition, TP antagonists that comprise an NO donor moiety are also contemplated.

TP antagonists also include polypeptides and nucleic acids that bind to TPs and inhibit their activity. The TP modulator may be selective or mixed TP antagonists or TP inhibitors. The receptor can be a human receptor.

Other TP modulators and ADP receptor antagonists contemplated by the invention include, e.g., those described in U.S. Pat. Nos. 6,689,786 and 7,056,926, U.S. patent application Ser. Nos. 11/556,490 and 11/556,518, and U.S. Provisional Patent Application No. 60/846,328, and pharmaceutically acceptable salts thereof.

2. ADP Receptor Modulators

The term “ADP receptor antagonist” as used herein refers to a compound that can inhibit or reduce the activity of an ADP receptor by at least about 30%, 40%, 50%, 55%, 60%, 65%, 70%, 75%, 80%, 85%, 90%, 95%, 96%, 97%, 98%, 99%, or 100% when used in therapeutically effective doses or concentrations. ADP receptor antagonists include small molecules and/or prodrugs including thienopyridine derivatives such as, e.g., clopidrogel. ADP receptor antagonists also include polypeptides and nucleic acids that bind to ADP receptors and inhibit their activity. An ADP receptor inactivator is an agent that modifies the receptor so as to block its activity. ADP receptor antagonists can include antibodies to the receptor. The antibodies may be monoclonal. They may be human or humanized antibodies. They may be directed to a human ADP receptor.

Examples of ADP receptor antagonists include, but are not limited to, thienopyridine derivatives such as clopidogrel, prasugrel, and ticlopidine, and direct acting agents such as cangrelor and AZD6140.

Examples of the ADP receptor modulators for use according to the instant invention include: 5-[(2-chlorophenyl)methyl]-4,5,6,7-tetrahydrothieno[3,2-c]pyridine described in U.S. Pat. No. 4,051,141 or U.S. Pat. No. 4,127,580; N-[2-(methylthio)ethyl]-2-[(3,3,3-trifluoropropyl)thio]-5′-adenylic acid, monoanhydride with dichloromethylenebisphosphonic acid described in U.S. Pat. No. 5,955,447 and Journal of Medicinal Chemistry, 1999, Vol. 42, p. 213-220; 2-(propylthio)-5′-adenylic acid, monoanhydride with dichloromethylenebis(phosphonic acid) described in Journal of Medicinal Chemistry, 1999, Vol. 42, p. 213-220; methyl(+)-(S)-α-(2-chlorophenyl)-6,7-dihydrothieno[3,2-c]pyridine-5(4H)-acetate described in U.S. Pat. No. 4,529,596, U.S. Pat. No. 4,847,265 or U.S. Pat. No. 5,576,328; 2-acetoxy-5-(α-cyclopropylcarbonyl-2-fluorobenzyl)-4,5,6,7-tetrahydrothieno[3,2-c]pyridine, or pharmaceutically acceptable salts thereof described in U.S. Pat. No. 5,288,726 or WO 02/04461; 5-[(2-chlorophenyl)methyl]-4,5,6,7-tetrahydrothieno[3,2-c]pyridine (particularly, its hydrochloride), N-[2-(methylthio)ethyl]-2-[-(3,3,3-trifluoropropyl)thio]-5′-adenylic acid, monoanhydride with dichloromethylenebisphosphonic acid, methyl(+)-(S)-α-(2-chlorophenyl)-6,7-dihydrothieno[3,2-c]pyridine-5(4H)-acetate (particularly, its sulfate), or 2-acetoxy-5-(α-cyclopropylcarbonyl-2-fluorobenzyl)-4,5-, 6,7-tetrahydrothieno[3,2-c]pyridine (particularly, its hydrochloride), or pharmaceutically acceptable salts thereof, more preferably, 2-acetoxy-5-(α-cyclopropylcarbonyl-2-fluorobenzyl)-4,5,6,7-tetrahydrothieno[3,2-c]pyridine, or pharmaceutically acceptable salts (particularly, its hydrochloride) thereof, and any other ADP modulators and ADP receptor antagonists described in these patents and applications.

In one embodiment, the ADP receptor antagonist has the structure shown in Formula I:

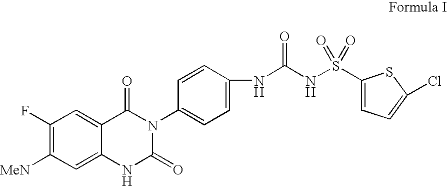

or is a pharmaceutically acceptable salt thereof. This compound is a reversible inhibitor of ADP-mediated platelet aggregation, which binds specifically to P₂Y₁₂ ADP receptor and has superior pharmacokinetic properties to clopidogrel. In addition, it has been demonstrated to de-aggregate preformed thrombi.

Additional and related antithrombotic agents are described, e.g., in U.S. Pat. Nos. 6,689,786, 7,022,731, 6,906,063, 7,056,926, 6,667,306, 6,762,029, 6,844,367, 6,376,515, 6,835,739, 7,022,695, 6,211,154 6,545,054, 6,777,413, 6,534,535, 6,545,055, 6,638,980, 6,720,317, 6,686,368, 6,632,815, 6,673,817, and 7,022,695, and U.S. patent application Ser. Nos. 11/304,054, 11/107,324, 11/236,051, 10/942,733, 10/959,909, 11/158,274, 11/298,317, 11/298,296, and 11/284,805. These agents may be purchased commercially or manufactured according to published methods.

In particular embodiments, the ADP modulator is an antagonist or inactivator of the platelet ADP receptor or a modulator of human CD39 (e.g., recombinant soluble ecto-ADPase/CD39).

ADP receptor modulators can be easily prepared according to the methods described, e.g., in U.S. Pat. No. 4,051,141, U.S. Pat. No. 4,127,580, U.S. Pat. No. 5,955,447, Journal of Medicinal Chemistry, 1999, Vol. 42, p. 213-220, U.S. Pat. No. 5,721,219, U.S. Pat. No. 4,529,596, U.S. Pat. No. 4,847,265, U.S. Pat. No. 5,576,328, U.S. Pat. No. 5,288,726 or WO 02/04461 or the analogous methods thereto (see also U.S. Patent Application Publication No. 20050192245 which is incorporated herein by reference as to the ADP modulator subject matter disclosed therein).

3. HMG CoA Reductase Inhibitors

HMG CoA reductase inhibitors, also referred to as statins, suitable for use herein include, but are not limited to, mevastatin and related compounds as disclosed in U.S. Pat. No. 3,983,140, lovastatin (mevinolin) and related compounds as disclosed in U.S. Pat. No. 4,231,938, pravastatin and related compounds such as disclosed in U.S. Pat. No. 4,346,227, simvastatin and related compounds as disclosed in U.S. Pat. Nos. 4,448,784 and 4,450,171, with pravastatin, lovastatin or simvastatin being preferred. Other HMG CoA reductase inhibitors which may be employed herein include, but are not limited to, fluvastatin, cerivastatin, atorvastatin, pyrazole analogs of mevalonolactone derivatives as disclosed in U.S. Pat. No. 4,613,610, indene analogs of mevalonolactone derivatives as disclosed in PCT application WO 86/03488, 6-[2-(substituted-pyrrol-1-yl)alkyl]pyran-2-ones and derivatives thereof as disclosed in U.S. Pat. No. 4,647,576, Searle's SC-45355 (a 3-substituted pentanedioic acid derivative) dichloroacetate, imidazole analogs of mevalonolactone as disclosed in PCT application WO 86/07054,3-carboxy-2-hydroxy-propane-phosphonic acid derivatives as disclosed in French Patent No. 2,596,393, 2,3-di-substituted pyrrole, furan and thiophene derivatives as disclosed in European Patent Application No. 0221025, naphthyl analogs of mevalonolactone as disclosed in U.S. Pat. No. 4,686,237, octahydro-naphthalenes such as disclosed in U.S. Pat. No. 4,499,289, keto analogs of mevinolin (lovastatin) as disclosed in European Patent Application No. 0,142,146 A2, as well as other known HMG CoA reductase inhibitors. In addition, phosphinic acid compounds useful in inhibiting HMG CoA reductase suitable for use herein are disclosed in GB 2205837.

4. Methods of Identifying Aspirin Sensitive, Intolerant, and Resistant Individuals

Methods of identifying individuals who are aspirin-sensitive, aspirin-intolerant, or aspirin-resistant are well known to ones of ordinary skill in the art (see, Jenkins et al., BMJ 328:434 (2004); Cowburn A. S., et al., J. Clin. Invest. 101(4):834-846 (1998); EP Patent Application Publication No. EP 1 190 714 A2; Nizankowska et al., Eur. Respir. J. 15:863-869 (2000); Casadevall J., et al., Thorax 55:921-24 (2000); Johnston et al., Eur. Respir. J. 8:411-15 (1995); and Ramanuja S., et al., Circulation 110:e1-e4 (2004)).

In addition, as aspirin-sensitive individual may be identified by reviewing the individual's medical records or querying the individual regarding whether they have previously had an adverse affect in response to aspiring or any other COX-1 inhibitor, or an NSAID, e.g., aspirin (Bayer®), ibuprofen (Advil®), naproxen (Aleve® or Naprosyn®), celecoxib (Celebrex®), diclofenac (Voltaren®), etodolac (Lodine®), fenoprofen (Nalfon®), indomethacin (Indocin®), ketoprofen, (Orudis®, Oruvail®), ketoralac (Toradol®), nabumetone (Relafen®), oxaprozin (Daypro®), sulindac (Clinoril®)), tolmetin (Tolectin®), and rofecoxib (Vioxx®).

Examples of typical adverse effects in response to aspirin include, e.g., decreased forced expiratory volume, decreased nasal volume, asthma, nausea, gastric bleeding, tinnitus, nasal congestion, cough, urticaria, and a drop in blood pressure.

Aspirin-sensitive individuals may also be identified bases upon their having an elevated level of leukotriene E4, which may be assayed, e.g., from a biological sample, such as blood or urine.

Methods of identifying aspirin-resistant individuals are described, e.g., in U.S. Patent Application Publication No. US2006/0160165. Several laboratory tests of platelet function have been designed and are available to identify aspirin-resistance using whole blood. The two main tools utilized are the Ultegra Rapid Platelet Function Assay (RPFA-ASA), and the PFA-100 device.

The RPFA-ASA cartridge has been specifically designed to address the level of inhibition of platelet aggregation achieved by aspirin treatment. As mentioned by the manufacturer, it is a qualitative measure of the effects of aspirin. In that assay, fibrinogen-coated beads agglutinate platelets through binding to GP IIb-IIIa receptors following stimulation by metallic cations and propyl gallate. The change in optical signal triggered by the agglutination (light transmittance increases as activated platelets bind and agglutinate the beads in the whole blood suspension) is measured. A recent study has detected a high incidence (23%) of aspirin non-responsiveness using this device, and determined a history of coronary artery disease to be associated with twice the odds of being an aspirin non-responder (Wang, J. C. et al., Am J Cardiol, 92(12):1492-4 (2003)). Aspirin resistance cannot be evaluated by the RPFA assay, however, in patients who were prescribed either GP IIb-IIIa inhibitors, dipyridamole, plavix (or ticlid), or NSAIDS (ibuprofen, naproxen, diclofenac, indomethacin, piroxicam), since those compounds interfere with the assay.

In the PFA-100 device, the platelet hemostatic capacity (PHC) of a citrated blood sample is determined by the time required for a platelet plug to occlude a 150 .mu.M aperture cut into a collagen-epinephrine coated membrane (used for the detection of aspirin). In the PFA-100 system, samples of citrated blood are aspirated through the aperture at shear rates of .about.4,000-5,000/sec. Under these high conditions of shear, vWF interactions with both GP Iba and GP IIb-IIIa trigger the thrombotic process. In the context of clinical events, plasma levels of vWF are expected to increase following platelet-rich thrombi formation and endothelial cell injury.

### B. Pharmaceutical Compositions

The TP modulator and other agents, including, e.g., ADP receptor modulators and HMG CoA Reductase inhibitors, maybe administered by any suitable route of deliver, including, e.g., orally, intranasally, rectally, sublingually, buccally, parenterally, or transdermally, and these agents may, thus, be formulated accordingly.

The agents are typically formulated with carriers, vehicles and/or excipients commonly employed in pharmaceutical compositions, e.g., talc, gum arabic, lactose, starch, magnesium stearate, cocoa bufter, aqueous or non-aqueous solvents, oils, paraffin derivatives, glycols, etc. Coloring and flavoring agents may also be added to preparations designed for oral administration.

Solutions may be prepared using water or physiologically compatible organic solvents such as ethanol, 1-2 propylene glycol, polyglycols, dimethyl sulfoxide, fatty alcohols, triglycerides, partial esters of glycerin, and the like. Parenteral compositions containing active ingredients may be prepared using conventional techniques and include sterile isotonic saline, water, 1,3-butanediol, ethanol, 1,2-propylene glycol, polyglycols mixed with water, Ringer's solution, etc.

The compositions may comprise one or more TP modulator and, optionally, one or more ADP modulators for use according to the invention. They may also further comprise one or more additional agents, such as, e.g., statins. These compounds may be formulated as their pharmaceutically acceptable salts. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate. Base salts include ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

Furthermore, any basic nitrogen-containing groups may be quaternized with agents like lower alkyl halides, such as methyl, ethyl, propyl and butyl chlorides, bromides and iodides; dialkyl sulfates, such as dimethyl, diethyl, dibutyl and diamyl sulfates, long chain halides, such as decyl, lauryl, myristyl and stearyl chlorides, bromides and iodides; aralkyl halides, such as benzyl and phenethyl bromides and others. Water or oil-soluble or dispersible products are thereby obtained.

The pharmaceutical compositions of the invention can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain stabilizers, pH modifiers, surfactants, bioavailability modifiers and combinations of these.

Pharmaceutical formulations may be prepared as liquid suspensions or solutions using a sterile liquid, such as oil, water, alcohol, and combinations thereof. Pharmaceutically suitable surfactants, suspending agents or emulsifying agents, may be added for oral or parenteral administration. Suspensions may include oils, such as peanut oil, sesame oil, cottonseed oil, corn oil and olive oil. Suspension preparation may also contain esters of fatty acids, such as ethyl oleate, isopropyl myristate, fatty acid glycerides and acetylated fatty acid glycerides. Suspension formulations may include alcohols, such as ethanol, isopropyl alcohol, hexadecyl alcohol, glycerol and propylene glycol. Ethers, such as poly(ethyleneglycol), petroleum hydrocarbons, such as mineral oil and petrolatum, and water may also be used in suspension formulations.

Pharmaceutically acceptable carriers that may be used in these compositions include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

According to one embodiment, the compositions of this invention are formulated for pharmaceutical administration to a mammal, preferably a human being. Such pharmaceutical compositions of the invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally or intravenously. The formulations of the invention may be designed as short-acting, fast-releasing, or long-acting. Still further, compounds can be administered in a local rather than systemic means, such as administration (e.g., injection) as a sustained release formulation.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. Compounds may be formulated for parenteral administration by injection such as by bolus injection or continuous infusion. A unit dosage form for injection may be in ampoules or in multi-dose containers.

The pharmaceutical compositions of this invention may be in any orally acceptable dosage form, including capsules, tablets, aqueous suspensions or solutions. In the case of tablets for oral use, carriers that are commonly used include lactose and corn starch. Lubricating agents, such as magnesium stearate, are also typically added. For a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be in the form of suppositories for rectal administration. These may be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be in a topical form, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract may be effected in a rectal suppository formulation (see above) or in a suitable enema formulation. Topically-transdermal patches may also be used. For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions may be formulated in a suitable lotion or cream containing the active components suspended or dissolved in one or more pharmaceutically acceptable carriers. Suitable carriers include mineral oil, sorbitan monostearate, polysorbate 60, cetyl esters, wax, cetyl alcohol, 2-octyidodecanol, benzyl alcohol and water.

The present invention provides dosage forms comprising a TP modulator, alone or in combination with an ADP modulator. These dosage forms may further comprise a statin. Dosage forms of the present invention comprise an amount of TP modulator, and any other agent present, sufficient to be effective when administered or taken in the prescribed amount. Thus, a dosage form may comprise a therapeutically effective amount of TP modulator, and any other agent present, in a single unit dosage form, e.g., tablet, or in two or more unit dosage forms, e.g., tablets. While it is desirous to include a therapeutically effective amount in a single unit dosage form, it is sometimes necessary to use two or more unit dosage forms to deliver a therapeutically effective amount, e.g., due to the volume of agent required.

Dosage forms may include, e.g., tablets, trochees, capsules, caplets, dragees, lozenges, parenterals, liquids, powders, and formulations designed for implantation or administration to the surface of the skin. Particularly suitable dosage forms are tablets or capsules for oral administration, as well as containers holding an intravenous loading dose. All dosage forms may be prepared using methods that are standard in the art (see, e.g., Remington's Pharmaceutical Sciences, 16th ed. A. Oslo. ed., Easton, Pa. (1980)).

Any of the above dosage forms containing effective amounts are within the bounds of routine experimentation and within the scope of the invention. A therapeutically effective dose may vary depending upon the route of administration and dosage form. The preferred compound or compounds of the invention is a formulation that exhibits a high therapeutic index. The therapeutic index is the dose ratio between toxic and therapeutic effects which can be expressed as the ratio between LD₅₀ and ED₅₀. The LD₅₀ is the dose lethal to 50% of the population and the ED₅₀ is the dose therapeutically effective in 50% of the population. The LD₅₀ and ED₅₀ are determined by standard pharmaceutical procedures in animal cell cultures or experimental animals.

The compositions described herein may be presented in unit-dose or multi-dose containers, such as sealed ampoules or vials. Such containers are typically sealed in such a way to preserve the sterility and stability of the formulation until use. In general, liquid formulations may be stored as suspensions, solutions or emulsions in oily or aqueous vehicles. Alternatively, a composition may be stored in a freeze-dried condition requiring only the addition of a sterile liquid carrier immediately prior to use.

Compositions for administration to a patient may take the form of one or more dosage units, where for example, a tablet, capsule or cachet may be a single dosage unit, and a container of a TP modulator in aerosol form may hold a plurality of dosage units. In certain embodiments, a dosage unit of a composition of the present invention is provided as a capsule or container holding an intravenous loading dose, comprising a formulation of a TP modulator suitable for intravenous administration in a therapeutically effective amount. In particular embodiments, a composition comprising an antithrombotic agent, such as a TP antagonist, is administered in one or more intravenous doses or by continuous infusion. In particular embodiments, an intravenous loading dose comprises about 1, 5, 10, 20, 30, 50, 100, 125, 150, 175, 200, 225, 250, 275, 300, 350, 400, 450, 500, 550, 600, 650, 700, 750, 800, 850, 900, 950, or 1000 mg of an antithrombotic agent, or a TP antagonist, such as ifetroban. In particular embodiments, intravenous loading dose comprises about 400-500 mg of ifetroban.

In particular embodiments, a composition comprising a TP antagonist is administered in one or more doses of a tablet formulation, typically for oral administration. The tablet formulation may be, e.g., an immediate release formulation, a controlled release formulation, or an extended release formulation. In particular embodiments, a tablet comprises about 1, 5, 10, 20, 30, 50, 100, 125, 150, 175, 200, 225, 250, 275, 300, 350, 400, 450, 500, 550, 600, 650, 700, 750, 800, 850, 900, 950, or 1000 mg of a TP antagonist, such as ifetroban. In particular embodiments, a tablet formulation comprises about 200-250 or 400-500 mg of ifetroban.

As used herein, “controlled release” refers to the release of the active ingredient from the formulation in a sustained and regulated manner over a longer period of time than an immediate release formulation containing the same amount of the active ingredient would release during the same time period. For example, an immediate release formulation comprising an antithrombotic agent may release 80% of the active ingredient from the formulation within 15 minutes of administration to a human subject, whereas an extended release formulation of the invention comprising the same amount of an antithrombotic agent would release 80% of the active ingredient within a period of time longer than 15 minutes, preferably within 6 to 12 hours. Controlled release formulations allows for less frequency of dosing to the mammal in need thereof. In addition, controlled release formulations may improve the pharmacokinetic or toxicity profile of the compound upon administration to the mammal in need thereof.

As used herein, “extended release” refers to the release of the active ingredient from the formulation in a sustained and regulated manner over a longer period of time than an immediate release formulation containing the same amount of the active ingredient would release during the same time period. For example, an immediate release formulation comprising an antithrombotic agent may release 80% of the active ingredient from the formulation within 15 minutes of administration to a human subject, whereas an extended release formulation of the invention comprising the same amount of antithrombotic agent would release 80% of the active ingredient within a period of time longer than 15 minutes, preferably within a period of time longer than 12 hours, e.g., 24 hours. Furthermore, the extended release formulations of the invention release the active ingredient, preferably ifetroban, over a longer period of time in vivo than a comparative controlled release formulation containing the same amount of the active ingredient would over the same period of time. As a non-limiting example, a comparative controlled release formulation containing the active ingredient, ifetroban, may release 80% of the amount of the active ingredient present in the formulation in vivo over a period of 4-6 hours after administration to a human subject, whereas an extended release formulation of the invention may release 80% of the same amount of the active ingredient in vivo over a period of 6-24 hours. Extended release formulations of the invention therefore allow for less frequency of dosing to the patient than the corresponding controlled release formulations. In addition, extended release formulations may improve the pharmacokinetic or toxicity profile of the active ingredient upon administration to the patient.

These unit dose formulations may be prepared for administration to a patient once a day, twice a day, or more than twice a day. The desired dose of the pharmaceutical composition according to this invention may conveniently be presented in a single dose or as divided dose administered at appropriate intervals, for example as two, three or more doses per day. In certain embodiments, a suitable daily dose of a TP modulator for an adult is between 1 and 5000 mg, between 1 and 1000 mg, between 10 and 1000 mg, between 50 and 500 mg, between 100 and 500 mg, between 200 and 500 mg, between 300 and 500 mg, or between 400 and 500 mg per day. Accordingly, when administered twice daily, a suitable single dose for an adult is between 0.5 and 2500 mg, between 0.5 and 500 mg, between 5 and 500 mg, between 25 and 250 mg, between 50 and 250 mg, between 100 and 250 mg, between 150 and 250 mg, or between 200 and 250 mg. Unit dose formulations may be readily adapted for multi-dosing.

In particular embodiments, a unit dosage form of ifetroban is a single capsule containing about 450 mg of ifetroban, or two capsules, each containing about 225 mg of ifetroban.

Besides those representative dosage forms described above, pharmaceutically acceptable excipients and carriers and dosage forms are generally known to those skilled in the art and are included in the invention. It should be understood that a specific dosage and treatment regimen for any particular patient will depend upon a variety of factors, including the activity of the specific compound employed, the age, body weight, general health, sex and diet of the patient, and the time of administration, rate of excretion, drug combination, judgment of the treating physician and severity of the particular disease being treated. The amount of active ingredient(s) will also depend upon the particular compound and other therapeutic agent, if present, in the composition.

As described herein, an antithrombotic agent of the present invention, e.g., a TP antagonist, may be used in combination with one or more other antithrombotic agents or pharmaceutical agents, including, e.g., a TP antagonist, a thromboxane antagonist, an ADP receptor antagonist, or a CD39 modulato. When used in combination, it is understood that lower dosages of one or more of the combined antithrombotic agents may be utilized to achieve a desired effect, since the two or more antithrombotic agents may act additively or synergistically. Accordingly, a therapeutically effective dosage of one or more combined antithrombotic agents may correspond to less than 90%, less than 80%, less than 70%, less than 60%, less than 50%, less than 40%, less than 30% or less than 20% of the therapeutically effective dosage when the antithrombotic agent is administered alone.

The two or more antithrombotic agents may be administered at the same time or at different times, by the same route of administration or by different routes of administration. For example, in order to regulate the dosage schedule, the antithrombotic agents may be administered separately in individual dosage units at the same time or different coordinated times. The respective substances can be individually formulated in separate unit dosage forms in a manner similar to that described above. However, fixed combinations of the antithrombotic agents are more convenient and are preferred, especially in tablet or capsule form for oral administration.

Thus, the present invention also provides unit dose formulations comprising two or more antithrombotic agents, wherein each thrombotic agent is present in a therapeutically effective amount when administered in the combination.

In particular embodiments, a patient is provided with ifetroban and one or more additional antithrombotic agents. In addition, the present invention includes a combination unit dose formulation comprises ifetroban and one or more addition antithrombotic agents. For example, methods of the present invention may comprise providing to a patient ifetroban in combination with another TP antagonist or an ADP receptor antagonist. In particular embodiments, ifetroban is provided in combination with a P2Y12 inhibitor, clopidogrel, prasugrel, or cangrelor. In particular embodiments, additional antithrombotic agents are provided (in combination with ifetroban or another antithrombotic agent) in an amount previously indicated as effective when the agent is used in combination with aspirin.

In certain embodiments, clopidogrel is provided in an oral daily dosage within the range from about 10 to about 1000 mg and preferably from about 25 to about 600 mg, and most preferably from about 50 to about 100 mg. In one particular embodiment, approximately 400-500 mg of ifetroban and approximately 50-100 mg of clopidogrel is provided to a patient per day. In a related embodiment, approximately 200-400 mg of ifetroban and 25-50 mg of clopidogrel is provided to a patient per day. In one particular embodiment, a patient is provided with about 450 mg of ifetroban and about 75 mg of clopiogrel (e.g., Plavix®) per day.

In certain embodiments, ticlopidine is provided in a daily dosage as set out in the 1997 PDR (250 mg bid) although daily dosages of from about 10 to about 1000 mg, preferably from about 25 to about 800 mg may be employed in accordance with the present invention. In one particular embodiment, approximately 400-500 mg of ifetroban and approximately 250-750 mg of ticlopidine is provided to a patient per day. In a related embodiment, approximately 200-400 mg of ifetroban and 100-250 mg of ticlopidine is provided to a patient per day. In one particular embodiment, a patient is provided with about 450 mg of ifetroban and about 500 mg of ticlopidine (e.g., Tidclid®) per day.

In certain embodiments, prasugrel is provided in a daily dosage of 1 to 100 mg per day, or about 10 mg per day. In one particular embodiment, approximately 400-500 mg of ifetroban and approximately 1 to 100 mg of prasugrel is provided to a patient per day. In a related embodiment, approximately 200-400 mg of ifetroban and 1 to 5 mg of prasugrel is provided to a patient per day. In one particular embodiment, a patient is provided with about 450 mg of ifetroban and about 10 mg of prasugrel per day.

The present invention further provides unit dosages comprising ifetroban and one or more additional antithrombotic agents, including any of those described herein. In particular embodiments, the additional antithrombotic agent is an ADP receptor antagonist. In particular embodiments, the additional antithrombotic agent is a P2Y12 inhibitor. Unit dosages of the present invention, in particular embodiments, comprise a daily dosage of ifetroban and a daily dosage of the additional one or more antithrombotic agents. Alternatively, a unit dosage comprises a portion of a daily dosage such as 50% of a daily dosage of the antithrombitic agents, so that the daily dosage may be taken in two unit dosages, e.g., at the same time or at different times.

The following examples are provided by way of illustration only and not by way of limitation. Those of skill will readily recognize a variety of non-critical parameters which could be changed or modified to yield essentially similar results.

### EXAMPLES

### Example 1

### Antithrombotic Activity of Ifetroban and Aspirin in Normal Volunteers

Ifetroban spiked (added in vitro) into the whole blood of normal volunteers (n=10) exhibits similar antithrombotic activity to aspirin (325 mg/day for a minimum of 5 days; FIG. 3). The experiment was performed by perfusing whole blood anticoagulated with factor Xa inhibitor (10 μM) through a human collagen type III-coated perfusion chamber. Platelets were fluorescently labeled in vitro using Rhodamine 6G (1.25 μg/ml final concentration), and thrombosis was monitored in real time by measuring the amount of fluorescent platelets recruited on the collagen surface using fluorescence microscopy. The results are depicted in FIG. 3.

### Example 2

### The Effects of an ADP Modulator

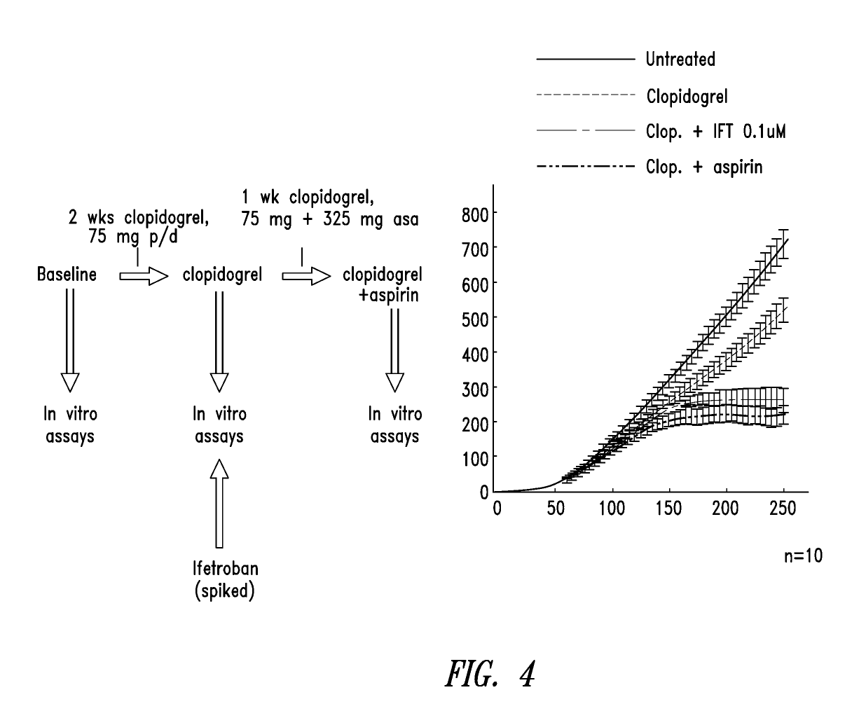

The effects of an ADP modulator were next investigated with respect to the antithrombotic effects of ifetroban spiked into the whole blood of normal volunteers (n=10) treated with clopidogrel (75 mg/day for 2 weeks), as observed using the perfusion chamber device. Ifetroban (at concentrations ranging from 30 nM to 1 μM) inhibited thrombosis to the same extent as aspirin on a clopidogrel background, as depicted in FIG. 4.

### Example 3

### Role of PGD2 in Destabilizing Thrombi

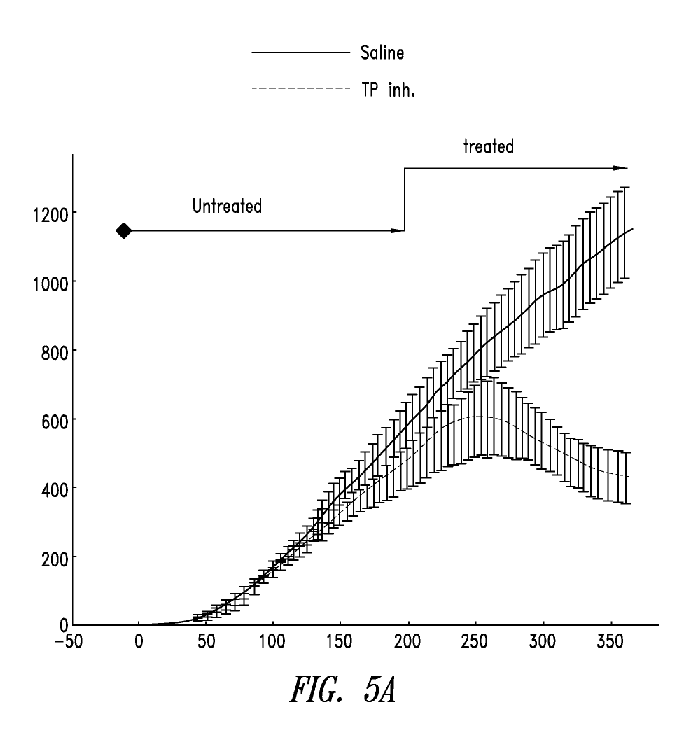

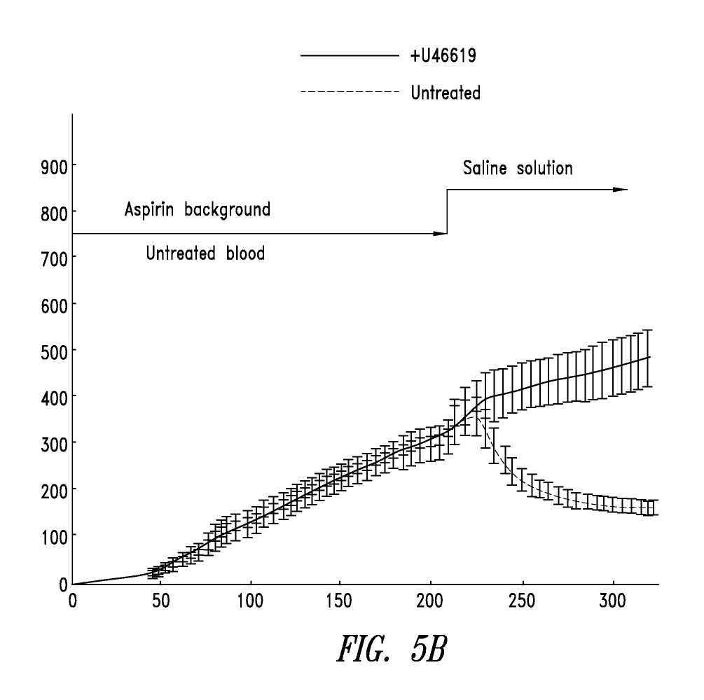

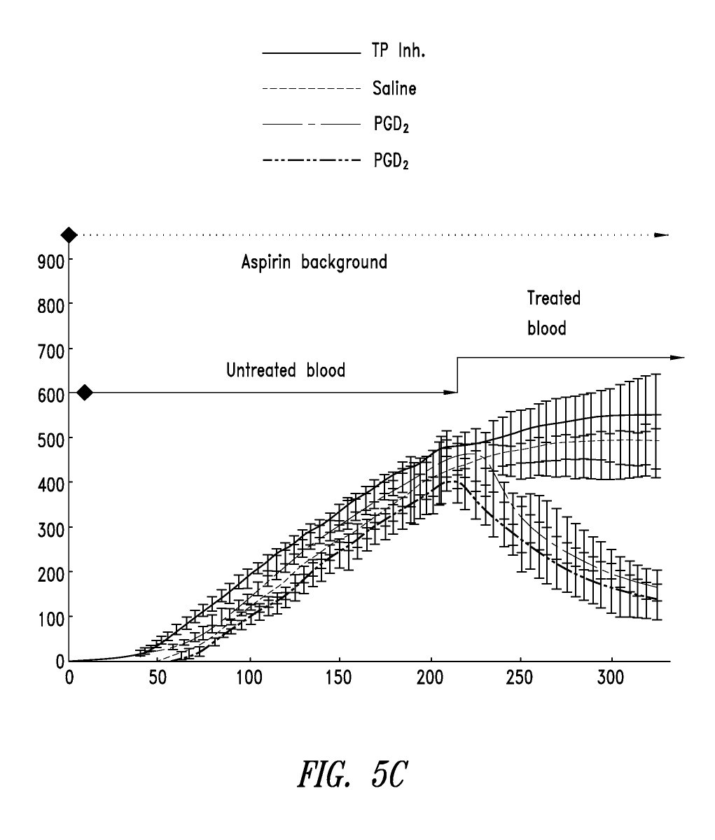

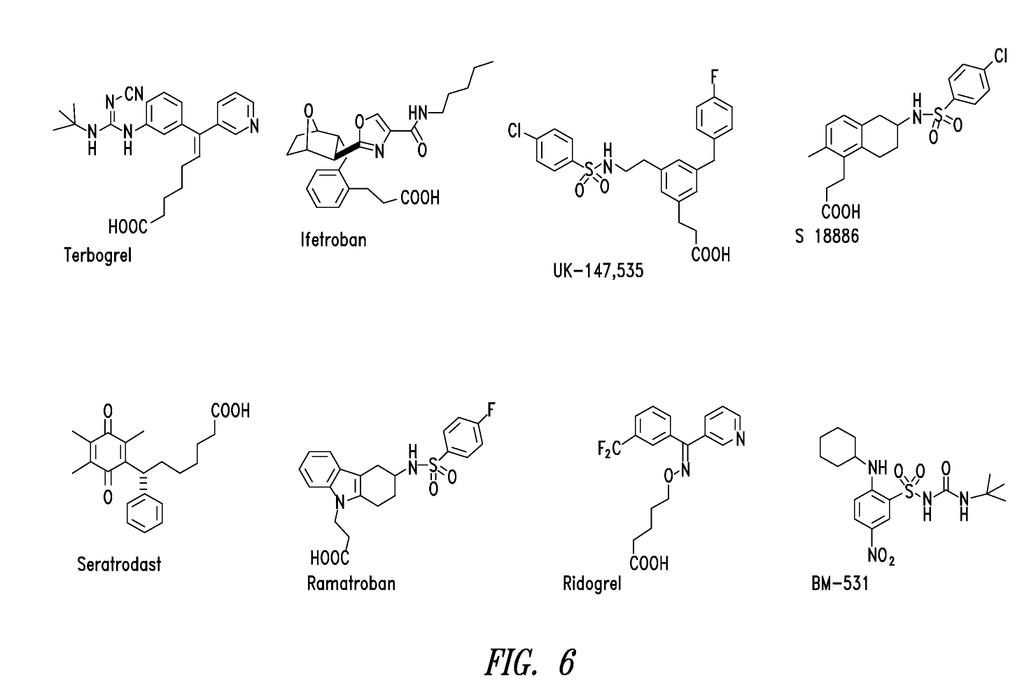

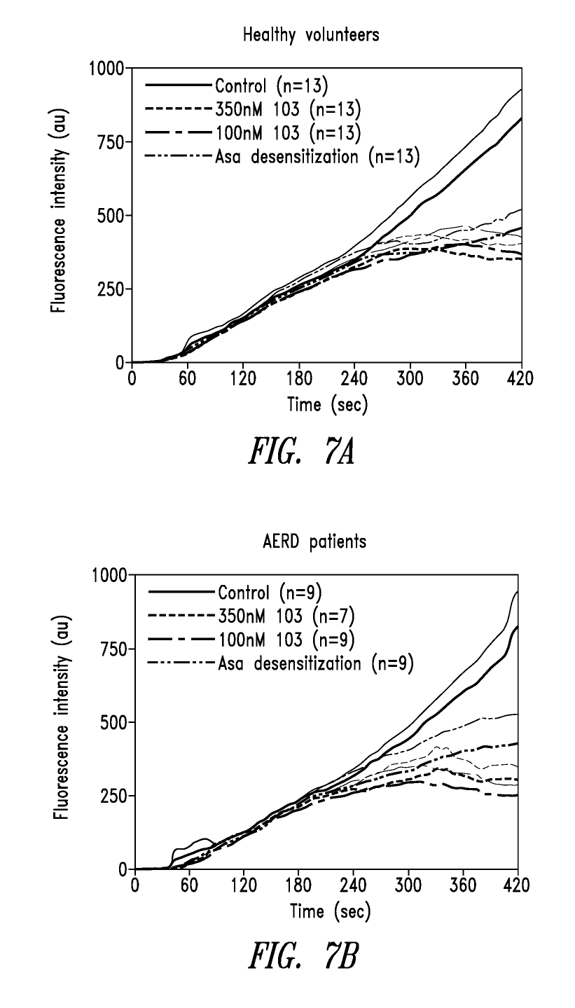

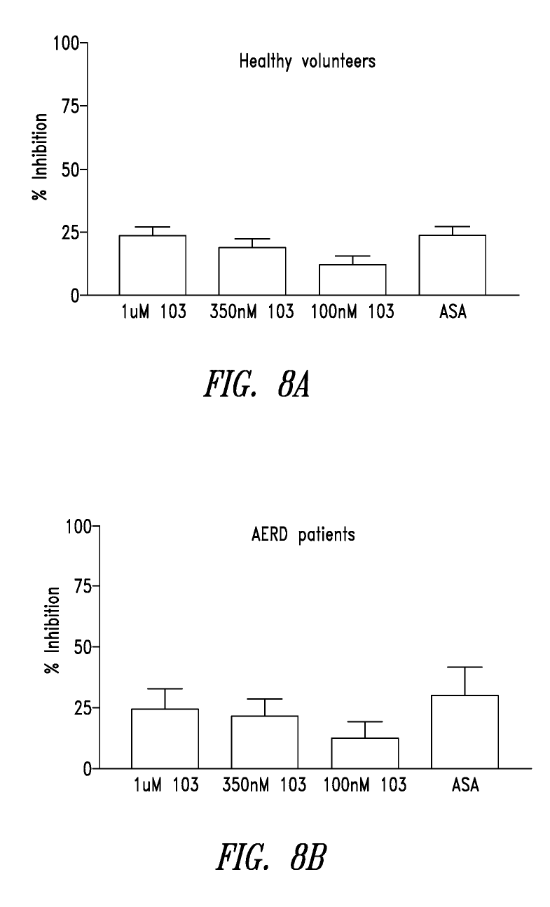

The addition of a TP antagonist to preformed thrombi caused dethrombosis (FIG. 5A). Since U46619 (TXA₂ mimetic) can promote thrombus stability (FIG. 5B), it was concluded that continued TXA₂-induced signaling through TP is required to maintain stable thrombi (FIG. 5B). It was also surprisingly found that the dethrombotic activity of a TP antagonist was prevented by prior aspirin treatment (FIG. 5C). Addition of physiological concentrations of PGD₂ induced a profound dethrombotic activity in aspirinated blood.

### Discussion of the Above Results

Previous in vivo studies have shown that COX-1 inhibition partially inhibits the antiplatelet activity achieved when the thromboxane pathway is blocked. Gresele and collaborators found that indomethacin blocked the ability of dazoxiben (a thromboxane synthase inhibitor) to promote the bleeding time prolongation activity of BM 13.177 (a thromboxane receptor antagonist) (see, Gresele P., et al., J. Clin. Invest. 80:1435-45 (1987)). Fitzgerald and coworkers showed that aspirin inhibited the prolongation in occlusion time in the coronary arteries of dogs achieved by the combined use of U63,557a (a thromboxane synthase inhibitor) with L636,499 (a thromboxane receptor antagonist) (see, Fitzgerald, D. J., et al., J. Clin. Invest. 82:1708-13 (1988)). It has been long recognized in the platelet literature, that while aspirin blocks the production of TXA₂, a prothrombotic mediator, it also decreases the activity of antithrombotic mediators such as PGD₂ (PGD₂ is one of the platelet inhibitory prostaglandins generated by COX-1). PGD₂ was now surprisingly discovered to be a potent endogenous dethrombosis agent (FIG. 5C) whose effects are not blocked by a TP modulator. The results described above indicate that dethrombosis activity achieved by TP antagonism is due to endogenous COX-1-dependent inhibitors of stability such as PGD₂. The mechanism by which PGD₂ effects platelet reactivity is known, and involves activation of the platelet adenylate cyclase (see, Cooper B., et al., Blood 54:684-93 (1979)), a pathway shared by inhibitors of P2Y₁₂. Thus these data predict reversal of thrombosis by TP antagonists.

The state-of-the-art chronic antiplatelet therapy utilized in patients with coronary artery disease consists in a combination of aspirin and clopidogrel. The antithrombotic profiles of 12 healthy individuals on clopidogrel (75 mg/d for 2 weeks) in presence of a direct TP antagonist (Ifetroban, spiked into the whole blood at the end of the 2^{nd} week) have been evaluated and compared to that obtained upon a combination therapy (75 mg/d clopidogrel for 3 weeks+325 mg/d aspirin over the third week). Experimental data indicate that both Ifetroban and aspirin effects synergized with the antithrombotic activity of clopidogrel and predict at least a non-inferiority of TP antagonist vs aspirin on a P2Y₁₂ antagonism background (FIG. 4).

The CLARITY study has shown some benefits associated with the use of clopidogrel (improvement of the patency rate of the infarct-related artery and reduction in ischemic complications) in acute myocardial infarction (AMI) patients with ST-segment elevation (see, Sabatine M. S., et al. NEJM 352:1179-89 (2005)). However, the inability of clopidogrel to reverse the shortened ST segment elevation may indicate that the active metabolite of clopidogrel (clopidogrel is a prodrug that requires hepatic metabolism to generate the active metabolite that blocks P2Y₁₂) failed to achieve the concentration required to induce dethrombosis. Therefore, it is expected that TP modulators with fast onset of action would provide higher protection to the clopidogrel treated AMI patients.

One main advantage of TP modulators or inhibitors of TXA₂ is that they do not affect the synthesis of endogenous negative modulators of thrombosis and inflammation. Indeed, inhibitors of TP and mixed synthase/TP antagonists do not reduce (and potentially increase) the PGD₂ and PGE₂ levels. It is therefore expected that aspirin-tolerant and aspirin-intolerant asthmatics patients with coronary artery disease will benefit from the protective effects of TP antagonists with no associated risks.

### Example 4

### Antithrombotic Effects of Indomethacin or Ifetroban in Combination with a P2Y₁₂ Antagonist

In this study, the antithrombotic and anti-aggregatory effects of indomethacin (between 10 nM and 10 uM), vehicle control and ifetroban (between 10 nM and 1 uM) spiked in vitro to whole blood containing a fixed concentration of a direct P2Y₁₂ antagonist are investigated. P2Y₁₂ is one of the 2 ADP receptors present on the surface of platelets that is targeted by the active metabolite of clopidogrel.

The experiments are performed in the real time thrombosis perfusion chamber device. In this system, the real time thrombotic process triggered by perfusion of Factor Xa anticoagulated whole blood is studied using a collagen coated perfusion chamber at arterial shear rates. Indomethacin and Ifetroban are expected to provide additional inhibition of the thrombotic process to that achieved by a direct P2Y₁₂ antagonist.

PRP-induced platelet aggregation are performed with arachidonic acid (between 200 μM and 1 mM), U46619 (a TP agonist) (between 0.5 μM and 5 μM) and collagen (around 4 μg/ml) as platelet agonists. Indomethacin and

Ifetroban are expected to provide additional inhibition of the aggregation process to that obtained with a direct P2Y12 antagonist for collagen-induced platelet aggregation.

### Example 5

### Antithrombotic Effect of Ifetroban and Aspirin in Combination with Clopidogrel In Vivo

The antithrombotic activity of ifetroban and aspirin is evaluated in vivo using WT and P2Y₁₂ heterozygous mice or WT mice treated with clopidogrel. The results will demonstrate in vivo that the combination of a P2Y₁₂ and TP antagonists can provide a similar protection against clot formation as a P2Y₁₂ antagonist and aspirin.

The methodology utilized is intravital microscopy. In this system, injury of the mesenteric arteries is performed by topical application of a filter paper that has been previously soaked into a ferric chloride solution. Monitoring of the recruitment of fluorescently labeled platelets on the injured vessel wall is performed in real time using an inverted fluorescent microscope connected to a computer. Ifetroban and aspirin are expected to display at least similar antithrombotic activity in wild type animals and profound inhibition in P2Y₁₂ heterozygous animals.

In vivo monitoring of gastrointestinal bleeding and determination of the levels of leukotrienes will be performed. It is expected that leukotrienes will be increased in response to aspirin therapy, while maintained constant in ifetroban-treated animals.

### Example 6

### Antithrombotic Effect of Indomethacin or Ifetroban in Combination with Clopidogrel in Aspirin-Intolerant Patients

A dose response study of the antithrombotic and anti-aggregatory effects of vehicle control, indomethacin (from 10 nM to 10 uM), and ifetroban (from 10 nM to 3 uM) spiked in vitro in the blood of aspirin-intolerant individuals before, and after treatment with clopidogrel (75 mg/d for a minimum of 5 days, n≧10) is performed. Experiments are performed in the real time perfusion chamber assay under arterial shear rates conditions. It is expected that ifetroban and indomethacin will increase the extent of inhibition achieved by clopidogrel. The goal of the study is to demonstrate that additional protection can be achieved in aspirin-intolerant individuals by using a TP antagonist. Indomethacin is a Cox-1 inhibitor that is used to replace aspirin in vitro (aspirin cannot be used in vitro for technical reasons or in vivo since aspirin intolerant asthmatic (AIA) individuals are evaluated). By using indomethacin we can confirm that Cox-1 inhibition indeed can provide additional antithrombotic properties; however, the intolerance prevents the use of a COX-1 inhibitor.

Collagen, AA, and U46619-induced platelet aggregation is performed as before. Increased inhibition versus clopidogrel alone is expected to be observed.

### Example 7

### Tolerability, Safety, and Pharmacokinetic Analysis of Ifetroban in Normal and Aspirin-Sensitive or Aspirin-Intolerant Patients

Tolerability, safety and PD assays are performed concomitantly. The anti-aggregatory and antithrombotic activity of ifetroban are assessed first in normal volunteers (at baseline and after treatment for 5 days) and then in aspirin-sensitive, aspirin-intolerant, and aspirin-resistant individuals. Doses of ifetroban to be tested/achieved in plasma range between 10 nM and 1 uM.

The thrombotic process is evaluated using the perfusion chamber system performed under arterial shear rates conditions in absence and presence of a fixed dose of a P2Y₁₂ antagonist at a dose showing equivalent antithrombotic activity than that of clopidogrel in normal volunteers [0.625-1.25 uM]). Evaluation of U46619-, AA- and collagen-induced platelet aggregation is conducted as above.

In further subjects, an ADP modulator is further administered.

Signs and symptoms of aspirin-intolerance are monitored. The TP modulator, alone, or in combination with the ADP modulator, does not induce the signs and symptoms of aspirin sensitivity or intolerance or resistance.

### Example 8

### Antiplatelet Effects of Ifetroban in Aspirin-Tolerant and Aspirin-Sensitive Patients

The thrombotic profile of aspirin intolerant (AERD)-asthmatic patients (AIA) patients and healthy volunteers was evaluated by comparing the antiplatelet effects of ifetroban and aspirin after desensitization using a physiological platelet agonist, essentially as described in Example 1.

Real time perfusion chamber assays (RTTP) were performed using blood anticoagulated with Fxa inhibitor (10 uM 034) and perfused through collagen-coated capillaries (1100/sec). Thrombus formation on the collagen surface was monitored in real time using fluorescence microscopy to detect fluorescently labeled (R6G) platelets.

Light transmittance aggregometry (LTA) assays were performed by standard procedures, initiating platelet aggregation with collagen or arachidonic acid.

Assays were performed pre- (+/−ifetroban, spiked in vitro) and post-aspirin desensitization.

As shown in FIG. 7, ifetroban had significant antithrombotic activity versus aspirin in both healthy volunteers (FIG. 7A) and AERD patients (FIG. 7B) when measured using the perfusion chamber assay.

Ifetroban also showed significant anti-aggregatory activity in both healthy volunteers and AERD patients in the collagen-induced platelet aggregation assay (FIG. 8). Specifically, ifetroban reproduced aspirin effects on collagen-induced platelet aggregation and thrombosis at concentrations >100 nM in both normal volunteers (FIG. 8A) and AERD patients (FIG. 8B).

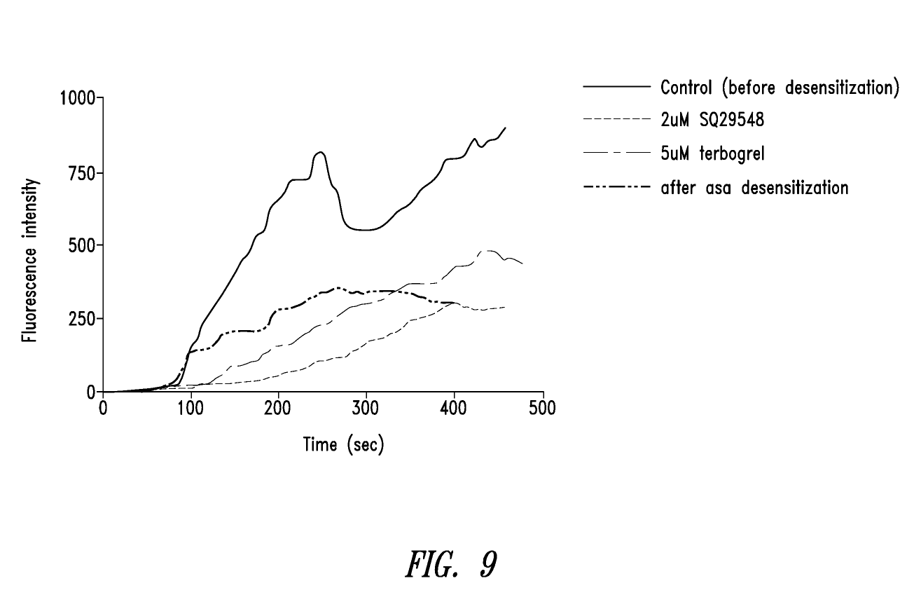

The ability of other TP antagonists to inhibit thrombosis was demonstrated using SQ29548, a direct acting TP antagonist, and terbogrel, a mixed inhibitor of TP and TxA synthase. As determined using the perfusion chamber test (RTTP), both SQ29548 and terbogrel spiked in vitro provided similar levels of inhibition of thrombosis as aspirin (after desensitization) in AERD patients (FIG. 9).

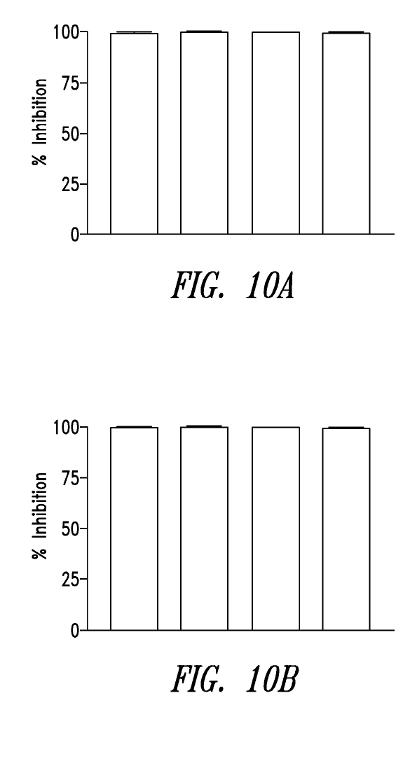

The anti-aggregatory activity of ifetroban versus aspirin in healthy volunteers and AERD patients was further demonstrated using an arachidonic acid-induced platelet aggregation assay (FIGS. 10A and 10B).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of treating or preventing a disease, disorder, or injury in an individual in whom therapy with a COX-1 inhibitor has proven to be harmful, or is predicted to be harmful, said method comprising administering to the individual a therapeutically effective amount of a TP modulator and, optionally, an ADP receptor modulator or a CD39 modulator.

2. The method of claim 1, wherein the COX-1 inhibitor is aspirin.

3. The method of claim 1, wherein the COX-1 inhibitor is a non-steroidal anti-inflammatory drug.

4. The method of claim 1, wherein the individual is aspirin-intolerant.

5. The method of claim 1, wherein the individual is aspirin-sensitive.

6. The method of claim 1, wherein the TP modulator is ifetroban, terbogrel, picotamide, S-18886, UK-147,535, seratodast-AA-2414, ramatroban, ridogrel, BMI-531, or a nitric oxide donating TP antagonist.

7. The method of claim 1, wherein the ADP receptor modulator is N-[2-(methylthio)ethyl]-2-[(3,3,3-trifluoropropyl)thio]-5′-adenylic acid, monoanhydride with dichloromethylenebisphosphonic acid, 2-(propylthio)-5′-adenylic acid, monoanhydride with dichloromethylene bis(phosphonic acid), methyl(+)-(S)-α-(2-chlorophenyl)-6,7-dihydrothieno[3,2-c]pyridine-5(4H)-acetate, or 2-acetoxy-5-(α-cyclopropylcarbonyl-2-fluorobenzyl)-4,5,6,7-tetrahydrothieno[3,2-c]pyridine.

8. The method of claim 1, wherein the ADP receptor modulator is clopidogrel.

9. The method of claim 1, wherein the effective amount of said TP modulator is from about 1 mg/kg to about 200 mg/kg.

10. The method of claim 1, wherein the effective amount of said TP modulator is from about 5 mg/kg to about 150 mg/kg.

11. The method of claim 1, wherein the effective amount of said TP modulator is from about 10 mg/kg to about 100 mg/kg.

12. The method of claim 1, wherein the effective amount of said TP modulator comprises from about 20 mg/kg to about 50 mg/kg.

13. The method of claim 1, further comprising the step of identifying the individual as being aspirin-sensitive or aspirin-intolerant prior to administering to the individual the therapeutically effective amount of the TP modulator and, optionally, the ADP receptor modulator or the CD39 modulator.

14. The method of claim 13, wherein the individual is queried to determine whether they have had a prior adverse reaction following administration of aspirin, wherein an affirmative response identifies the individual as being aspirin-sensitive.

15. The method of claim 14, wherein the adverse reaction is selected from the group consisting of: decreased forced expiratory volume, asthma, nausea, gastric bleeding, tinnitus, nasal congestion, cough, urticaria, and a drop in blood pressure.

16. The method of claim 1, wherein the individual is identified as being aspirin-sensitive by:
administering aspirin to the individual; and
screening a biological sample from the individual for the presence of leukotriene E4 (LTE4), wherein the presence of LTE4 in the biological sample identifies the individual as being aspirin sensitive.

17. The method of claim 16, wherein the biological sample is blood or urine.

18. The method of claim 1, wherein the individual is determined to be aspirin-sensitive by:
administering aspirin to the individual; and
measuring the individual's forced expiratory volume (FEV₁), wherein a decreased FEV₁ identifies the individual as being aspirin-sensitive.

19. The method of claim 1, wherein the individual is determined to be aspirin-sensitive by:
administering aspirin to the individual; and
measuring the individual's nasal volume, wherein a decreased nasal volume identifies the individual as being aspirin sensitive.

20. The method of claim 1, wherein the administering leads to a mean plasma concentration of the TP modulator from about 10 to about 500 ng/ml about 2 to about 10 hours after administration.

21. The method of claim 1, wherein the TP modulator is a TP antagonist.

22. The method of claim 21, wherein said TP antagonist is Ifetroban.

23. The method of claim 1, wherein the disease or disorder is a cardiovascular disease or disorder.

24. The method of claim 23, wherein the cardiovascular disease or disorder is acute coronary syndrome or a thrombotic disorder.

25. The method of claim 24, wherein the acute coronary syndrome is selected from the group consisting of: acute myocardial ischemia, acute myocardial infarction, and angina.

26. The method of claim 24, wherein the thrombotic disorder is selected from the group consisting of: atherosclerosis, thrombocytosis, peripheral artery occlusion, and stenosis.

27. The method of claim 1, wherein the disease or disorder is selected from the group consisting of: sickle cell anemia, stroke, asthma, pulmonary hypertension, and acute lung injury.

28. The method of claim 1, comprising administering an effective dose of an ADP receptor modulator to the individual.

29. The method of claim 28, wherein the effective dose of said ADP receptor modulator is from about 1 mg/kg to about 200 mg/kg.

30. The method of claim 28, wherein the effective dose of said ADP receptor antagonist is from about 1 mg/kg to about 150 mg/kg.

31. The method of claim 28, wherein the effective dose of said ADP receptor modulator is from about 10 mg/kg to about 100 mg/kg.

32. The method of claim 28, wherein the effective dose of said ADP receptor modulator comprises from about 20 mg/kg to about 50 mg/kg.

33. The method of claim 28, wherein the ADP receptor modulator is a thienopyridine derivative.

34. The method of claim 33, wherein the thienopyridine derivative is ticlopidine or prasugrel.

35. The method of claim 28, wherein the effective dose of the TP modulator is reduced by administration of the ADP receptor modulator.

36. The method of claim 35, wherein the effective dose of the TP antagonist is reduced by at least about 25% by administration of the ADP receptor modulator.

37. The method of claim 35, wherein the effective dose of the TP modulator is reduced by at least about 50% in the presence of the ADP receptor modulator.

38. The method of claim 35, wherein the effective dose of the TP modulator is reduced by at least about 75% by the administration of the ADP receptor modulator.

39. The method of claim 1, wherein the individual has a coronary stent.

40. The method of claim 1, wherein the individual is undergoing or scheduled to undergo a coronary artery bypass surgery.

41. A method of treating an individual for a cardiovascular disorder, said method comprising administering a therapeutically effective amount of a TP modulator and, optionally, an ADP receptor modulator or a CD39 modulator, and instructing or advising the individual not to take aspirin or an NSAID.

42. The method of claim 41, wherein the individual has had an acute arterial thrombosis.

43. The method of claim 41, wherein the individual is not known to be aspirin-sensitive or aspirin intolerant.

44. A method of treating or preventing thrombosis in an individual in whom therapy with a COX-1 inhibitor has proven harmful, said method comprising administering to the individual a therapeutically effective amount of a TP modulator and, optionally, an ADP receptor modulator or a CD39 modulator

45. A method of reducing platelet loss or aggregation during on-pump coronary bypass surgery of an individual, comprising administering to the individual an effective amount of a TP modulator and, optionally, an ADP receptor modulator or a CD39 modulator prior to or during the coronary bypass surgery.

46. A method of inhibiting platelet aggregation in an aspirin-sensitive or aspirin-resistant individual, said method comprising administering to the individual an amount of ifetroban sufficient to maintain a blood concentration of at least 350 nM for at least 6, 12, 24, or 48 hours.

47. The method of claim 46, further comprising administering an ADP receptor antagonist to the individual.
